SEQUENCE LISTING (88 sequences)

SEQ ID NO: 1 - DNA, 354 bp, synthetic construct
  source 1..354: mol_type = other DNA
cagtcggtggaggagtccgggggtcgcctggtcacgcctgggacagccctgacactcacctgcacagtctctggattctccctcagtagctattcaatgggctgggtccgccaggctccagggaaggggctggagtggatcggatatcttaatactggtggtagcacatactacgcgagctgggtgaatggtcgattcaccatctccaaaacctcgtcgaccacggtggatctgaaaatcaccagtccgacaaccgaggacacggccacctatttctgtaccagggccgggggtgttagtagcaatatgggctttaacatctggggccaagggaccctggtcaccgtctcgagc

SEQ ID NO: 2 - protein, 118 aa, synthetic construct
QSVEESGGRLVTPGTPLTLTCTVSGIDLSGFAMGWVRQAPGKGPEYIGFIDSGGGAFYATWARGRFTISRTSSTTVDLKMTSLTAADTATYFCARHGGNTYYYAMDPWGPGTLVTVSS

SEQ ID NO: 3 - DNA, 330 bp, synthetic construct
  source 1..330: mol_type = other DNA
gcccttgtgatgacccagactccagcctccgtggaggcagctgtgagaggcacagtcaccatcaagtgccaggccagtcagagcattagtaactacttagcctggtatcagcagaaaccagggcagcctcccaagctcctgatctattctgcatccactctggcatctggggtctcatcgcggttcaaaggcagtggatctgggacacagttcactctcaccatcagcggcgtggagtgtgccgatgctgccacttactactgtcaacaggtgtatagtgtcactaatattgataatgctttcggcggaggcaccgaggtggagttcaaa

SEQ ID NO: 4 - protein, 110 aa, synthetic construct
ALVMTQTPASVEAAVRGTVTIKCQASQSISNYLAWYQQKPGQPPKLLIYSASTLASGVSSRFKGSGSGTQFTLTISGVECADAATYYCQQVYSVTNIDNAFGGGTEVEFK

SEQ ID NO: 5 - DNA, 357 bp, synthetic construct
  source 1..357: mol_type = other DNA
caggtgcagctgcaggagtccggcggcaggctgatcaagcccggcgagcccctgaggctgtcctgcaagacctccggcatcgacctgtccggcttcgccatgggctgggtgaggcaggcccccggcaagggcctggagtacatcggcttcatcgactccggcggcggcgccttctacgccacctgggccaggggcaggttcaccatctccaggacctccaccaacaccgtgtacctgcagatgaactccctgaccgccgaggacaccgccgtgtactactgcgccaggcacggcggcaacacctactactacgccatggacccctggggccccggcaccctggtgaccgtgtcctcc

SEQ ID NO: 6 - protein, 119 aa, synthetic construct
QVQLQESGGRLIKPGEPLRLSCKTSGIDLSGFAMGWVRQAPGKGLEYIGFIDSGGGAFYATWARGRFTISRTSTNTVYLQMNSLTAEDTAVYYCARHGGNTYYYAMDPWGPGTLVTVSS

SEQ ID NO: 7 - DNA, 330 bp, synthetic construct
  source 1..330: mol_type = other DNA
gccctgaggatgacccagtccccctcctccctggccgccaccaccggccagagggtgaccatcacctgccaggcctcccagtccatctccaactacctggcctggtaccagcagaagcccggccagccccccaagctgctgatctactccgcctccaccctggcctccggcgtgccctccaggttcaagggctccggctccggcacccagttcaccctgaccatctccagcgtgcagcccgaggacttcgccacctactactgccagcaggtgtactccgtgaccaacatcgacaacgccttcggcggcggcaccagggtggagatcaag

SEQ ID NO: 8 - protein, 143 aa, synthetic construct
SEQUENCEIDFBSMVLAMINOACIDSEQUENCEALRMTQSPSSLAATTGQRVTITCQASQSISNYLAWYQQKPGQPPKLLIYSASTLASGVPSRFKGSGSGTQFTLTISCVQCEDFATYYCQQVYSVTNIDNAFGGGTRVEIK

SEQ ID NO: 9 - DNA, 357 bp, synthetic construct
  source 1..357: mol_type = other DNA
caggtgcagctggtggagtccggcggcggcctggtgaagcccggcggctccctgaggctgtcctgcgccgcctccggcatcgacctgtccggcttcgccatgggctgggtgaggcaggcccccggcaagggcctggagtgggtgggcttcatcgactccggcggcggcgccttctacgccacctgggccaggggcaggttcaccatctccaggacctccaagaacaccgtgtacctgcagatgaactccctgagggccgaggacaccgccgtgtactactgcgccaggcacggcggcaacacctactactacgccatggacccctggggccagggcaccctggtgaccgtgtcctcc

SEQ ID NO: 10 - protein, 119 aa, synthetic construct
QVQLVESGGGLVKPGGSLRLSCAASGIDLSGFAMGWVRQAPGKGLEWVGFIDSGGGAFYATWARGRFTISRTSKNTVYLQMNSLRAEDTAVYYCARHGGNTYYYAMDPWGQGTLVTVSS

SEQ ID NO: 11 - DNA, 330 bp, synthetic construct
  source 1..330: mol_type = other DNA
gccctggtgatgacccagtccccctcctccctgtccgcctccgtgggcgacagggtgaccatctcctgccaggcctcccagtccatctccaactacctggcctggtaccagcagaagcccggccaggcccccaagctgctgatctactccgcctccaccctggcctccggcgtgccctccaggttctccggctccggctccggcaccgacttcaccctgaccatctcctccgtggagcccgaggacttcgccacctactactgccagcaggtgtactccgtgaccaacatcgacaacgccttcggcggcggcaccaaggtggagatcaag

SEQ ID NO: 12 - protein, 110 aa, synthetic construct
ALVMTQSPSSLSASVGDRVTISCQASQSISNYLAWYQQKPGQAPKLLIYSASTLASGVPSRFSGSGSGTDFTLTISSVEPEDFATYYCQQVYSVTNIDNAFGGGTKVEIK

SEQ ID NO: 13 - DNA, 333 bp, synthetic construct
  source 1..333: mol_type = other DNA
cagtcggtgaaggagtccgagggaggtctcttcaagccgacggataccctgacactcacctgcacagtctctggattctccctcagtacctatgcaatgagctgggtccgccaggctccagggaacgggctggaatggatcggaatcattgatgctgttggtcgcacatattacgcgagttgggcgaaaagtcgatccaccatcaccaggaacaccaacctgaacacggtgactctgaaaatgaccagtctgacagccgcggacacggccacctatttctgtgcgagatggtgggatctctggggccaaggcaccctggtcaccgtctcgagc

SEQ ID NO: 14 - protein, 111 aa, synthetic construct
QSVKESEGGLFKPTDTLTLTCTVSGFSLSTYAMSWVRQAPGNGLEWIGIIDAVGRTYYASWAKSRSTITRNTNLNTVTLKMTSLTAADTATYFCARWWDLWGQGTLVTVSS

SEQ ID NO: 15 - DNA, 330 bp, synthetic construct
  source 1..330: mol_type = other DNA
gcgcaagtgctgacccagactgcatcgtccgtgtctgccgctgtgggaggcactgtcaccatcaattgccagtccagtcagagtgtttatgatagcaacgacttaacctggtatcagcagaaaccagggcagtctcccaagctcctgatctatctggcatccactctggcatctggggtcccatcgcgattcaaaggcagtggatctgggacacagttcactctcaccatcagcgacctggagtgtgacgattctgccacttactactgtgcagcccattataatagtgatatttatactttcggcggagggaccgaggtggaggtcaaa

SEQ ID NO: 16 - protein, 110 aa, synthetic construct
AQVLTQTASSVSAAVGGTVTINCQSSQSVYDSNDLTWYQQKPGQSPKLLIYLASTLASGVPSRFKGSGSGTQFTLTISDLECDDSATYYCAAHYNSDIYTFGGGTEVEVK

SEQ ID NO: 17 - DNA, 568 bp, synthetic construct
  source 1..568: mol_type = other DNA
ttctctccacaggtgtccactcccaggtccaagtttaaacggatctctagcgaattcaagcttacgctcaccatggagactgggctgcgctggcttctcctggtcgctgtgctcaaaggtgtccagtgtcagtcggtggaggagtccgggggtcgcctgataacgcctggaggatccctgacactcacctgcaccgtctctggaatcgacctcagtagcaatggaatgagctgggtccgccagtctccagggaaggggctggaatacatcggattcattgatactgatggtagcgcatactccgcgagctgggcgaaaggccgattcaccatctccaaagcctcgtcgaccacggtggatctgaaaatgaccagtctgacaaccgaggacacggccacctatttctgtgccagagcctatgttagtggtagtactggttacaattttaacatctggggcccggggaccctggtcaccgtctcgagcgctagcaccaagggcccatcggtcttccccctggcaccctcctccaagagcacctctgggggcacagcggccctgggctgct

SEQ ID NO: 18 - protein, 189 aa, synthetic construct
FSPQVSTPRSKFKRISSEFKLTLTMETGLRWLLLVAVLKGVQCQSVEESGGRLITPGGSLTLTCTVSGIDLSSNGMSWVRQSPGKGLEYIGFIDTDGSAYSASWAKGRFTISKASSTTVDLKMTSLTTEDTATYFCARAYVSGSTGYNFNIWGPGTLVTVSSASTKGPSVFPLAPSSKSTSGGTAALGC

SEQ ID NO: 19 - DNA, 540 bp, synthetic construct
  source 1..540: mol_type = other DNA
ttctctccacaggtgtccactcccaggtccaagtttaaacggatctctagcgaattcaagcttcgaatcgacatggacacgagggcccccactcagctgctggggctcctgctgctctggctcccaggtgccagatgtgcattcgaattgacccagactccatcctccgtggaggcagctgtgggaggcacagtcaccatcaagtgccaggccagtcagagcattagtagctacttagcctggtatcagcagaaaccagggcagcctcccaagtccctgatctacaaggcatccactctggcatctggggtcccatcgcggttcaaaggcagtggatctgggacagagttcactctcaccatcagcgacctggagtgtgccgatgctgccacttactactgtcaacagggttatactattcgtaatattgataatgctttcggcggagggaccgaggtggagttcaaacgtacggtggctgcaccatctgtcttcatcttcccgccatctgatgagcagttgaaatctggaactgcctct

SEQ ID NO: 20 - protein, 180 aa, synthetic construct
FSPQVSTPRSKFKRISSEFKLRIDMDTRAPTQLLGLLLLWLPGARCAFELTQTPSSVEAAVGGTVTIKCQASQSISSYLAWYQQKPGQPPKSLIYKASTLASGVPSRFKGSGSGTEFTLTISDLECADAATYYCQQGYTIRNIDNAFGGGTEVEFKRTVAAPSVFIFPPSDEQLKSGTAS

SEQ ID NO: 21 - DNA, 360 bp, synthetic construct
  source 1..360: mol_type = other DNA
caggtgcagctggtggagtccggcggcaggctgatcaagcccggcggctccctgaggctgacctgcaagacctccggcatcgacctgtcctccaacggcatgtcctgggtgaggcagccccccggcaagggcctggagtacatcggcttcatcgacaccgacggctccgcctactccgcctcctgggccaagggcaggttcaccatctccaaggcctccatcaacaccgtgttcctgcagatgacctccctgaagtccgaggacaccgccatctactactgcgccagggcctacgtgtccggctccaccggctacaacttcaacatctggggccccggcaccctggtgaccgtgtcctcc

SEQ ID NO: 22 - protein, 120 aa, synthetic construct
QVQLVESGGRLIKPGGSLRLTCKTSGIDLSSNGMSWVRQPPGKGLEYIGFIDTDGSAYSASWAKGRFTISKASINTVFLQMTSLKSEDTAIYYCARAYVSGSTGYNFNIWGPGTLVTVSS

SEQ ID NO: 23 - DNA, 330 bp, synthetic construct
  source 1..330: mol_type = other DNA
gccttcaggctgacccagaccccctcctccttcgccgccaccctgggccagagggtgaccatcacctgccaggcctcccagtccatctcctcctacctggcctggtaccagcagaagcccggcaagccccccaagtccctgatctacaaggcctccaccctggcctccggcgtgccctccaggttcaagggctccggctccggcaccgacttcaccctgaccatctccagcctgcagcccgacgacttcgccacctactactgccagcagggctacaccatcaggaacatcgacaacgccttcggcggcggcaccagggtggagatcaag

SEQ ID NO: 24 - protein, 110 aa, synthetic construct
AFRLTQTPSSFAATLGQRVTITCQASQSISSYLAWYQQKPGKPPKSLIYKASTLASGVPSRFKGSGSGTDFTLTISSLQPDDFATYYCQQGYTIRNIDNAFGGGTRVEIK

SEQ ID NO: 25 - DNA, 360 bp, synthetic construct
  source 1..360: mol_type = other DNA
caggtgcagctggtggagtccggcggcggcctgatcaagcccggcggctccctgaggctgtcctgcgccgcctccggcatcgacctgtcctccaacggcatgtcctgggtgaggcaggcccccggcaagggcctggagtgggtgggcttcatcgacaccgacggctccgcctactccgcctcctgggccaagggcaggttcaccatctccaaggactccaagaacaccgtgtacctgcagatgaactccctgagggccgaggacaccgccgtgtactactgcgccagggcctacgtgtccggctccaccggctacaacttcaacatctggggccagggcaccctggtgaccgtgtcctcc

SEQ ID NO: 26 - protein, 120 aa, synthetic construct
QVQLVESGGGLIKPGGSLRLSCAASGIDLSSNGMSWVRQAPGKGLEWVGFIDTDGSAYSASWAKGRFTISKDSKNTVYLQMNSLRAEDTAVYYCARAYVSGSTGYNFNIWGQGTLVTVSS

SEQ ID NO: 27 - DNA, 330 bp, synthetic construct
  source 1..330: mol_type = other DNA
gccttcgtgctgacccagtccccctcctccctgtccgcctccgtgggcgacagggtgaccatctcctgccaggcctcccagtccatctcctcctacctggcctggtaccagcagaagcccggccaggcccccaagtccctgatctacaaggcctccaccctggcctccggcgtgccctccaggttctccggctccggctccggcaccgacttcaccctgaccatctcctccctggagcccgaggacttcgccacctactactgccagcagggctacaccatcaggaacatcgacaacgccttcggcggcggcaccaaggtggagatcaag

SEQ ID NO: 28 - protein, 110 aa, synthetic construct
AFVLTQSPSSLSASVGDRVTISCQASQSISSYLAWYQQKPGQAPKSLIYKASTLASGVPSRFSGSGSGTDFTLTISSLEPEDFATYYCQQGYTIRNIDNAFGGGTKVEIK

SEQ ID NO: 29 - DNA, 561 bp, synthetic construct
  source 1..561: mol_type = other DNA
ttctctccacaggtgtccactcccaggtccaagtttaaacggatctctagcgaattcaagcttacgctcaccatggagactgggctgcgctggcttctcctgnncgctgtgctcaaaggtgtccagtgtcagtcggtggaggagtccgggggtcgcctggtcacgcctgggacagccctgacactcacctgcacagtctctggattctccctcagtagctattcaatgggctgggtccgccaggctccagggaaggggctggagtggatcggatatcttaatactggtggtagcacatactacgcgagctgggtgaatggtcgattcaccatctccaaaacctcgtcgaccacggtggatctgaaaatcaccagtccgacaaccgaggacacggccacctatttctgtaccagggccgggggtgttagtagcaatatgggctttaacatctggggccaagggaccctggtcaccgtctcgagcgctagcaccaaggnnccatcggtcttccccctggcaccctcctccaagagcacctctgggggcacagcggccctgggc

SEQ ID NO: 30 - protein, 187 aa, synthetic construct
FSPQVSTPRSKFKRISSEFKLTLTMETGLRWLLLXAVLKGVQCQSVEESGGRLVTPGTALTLTCTVSGFSLSSYSMGWVRQAPGKGLEWIGYLNTGGSTYYASWVNGRFTISKTSSTTVDLKITSPTTEDTATYFCTRAGGVSSNMGFNIWGQGTLVTVSSASTKXPSVFPLAPSSKSTSGGTAALG

SEQ ID NO: 31 - DNA, 541 bp, synthetic construct
  source 1..541: mol_type = other DNA
ttctctccacaggtgtccactcccaggtccaagtttaaacggatctctagcgaattcaagcttcgaatcgacatggacacgagggcccccactcagctgctggggctcctgctgctctggctcccaggtgccagatgtgaccctgtgctgacccagactccagcctccgtgtctgagcctgtgggaggcacagtcaccatcaagtgccaggccagtcagagcattagtagctacttagcctggtatcagcagaaaccagggcagcctcccaagctcctgatctacagggcatccactctggcatctggggtcccatcgcggttcaaaggcagtggatctgggacacagttcactctcaccatcagcgacctggagtgtgccgatgctgccacttactactgtcaatgtactgattatggctacaattatcttggggctttcggcggagggaccgaggtggagttcaaacgtacggtggctgcaccatctgtcttcatcttcccgccatctgatgagcagttgaaatctggaactgcctctg

SEQ ID NO: 32 - protein, 180 aa, synthetic construct
FSPQVSTPRSKFKRISSEFKLRIDMDTRAPTQLLGLLLLWLPGARCDPVLTQTPASVSEPVGGTVTIKCQASQSISSYLAWYQQKPGQPPKLLIYRASTLASGVPSRFKGSGSGTQFTLTISDLECADAATYYCQCTDYGYNYLGAFGGGTEVEFKRTVAAPSVFIFPPSDEQLKSGTAS

SEQ ID NO: 33 - DNA, 357 bp, synthetic construct
  source 1..357: mol_type = other DNA
caggtgcagctgctggagtccggcggcaggctgatcaagcccggcgagtccctgaagctgacctgcaagacctccggcttctccctgtcctcctactccatgggctgggtgaggcaggcccccggcaagggcctggagtggatcggctacctgaacaccggcggctccacctactacgcctcctgggtgaacggcaggttcaccatctccaagacctccatctccaccgtgttcctgcagctgaggtcccccaggtccgaggacaccgccctgtactactgcaccagggccggcggcgtgtcctccaacatgggcttcaacatctggggccagggcaccctggtgaccgtgtcctcc

SEQ ID NO: 34 - protein, 119 aa, synthetic construct
QVQLLESGGRLIKPGESLKLTCKTSGFSLSSYSMGWVRQAPGKGLEWIGYLNTGGSTYYASWVNGRFTISKTSISTVFLQLRSPRSEDTALYYCTRAGGVSSNMGFNIWGQGTLVTVSS

SEQ ID NO: 35 - DNA, 330 bp, synthetic construct
  source 1..330: mol_type = other DNA
gaccccgtgctgacccagtccccctccttcctgtccctgaccgtgggccagagggtgaccatcaactgccaggcctcccagtccatctcctcctacctggcctggtaccagcagaagcccggccagccccccaagctgctgatctacagggcctccaccctggcctccggcgtgccctccaggttctccggctccggctccggcaccgagttcaccctgaccatctcctccctgcagtccgacgacttcgccacctactactgccagtgcaccgactacggctacaactacctgggcgccttcggcggcggcaccagggtggagatcaag

SEQ ID NO: 36 - protein, 110 aa, synthetic construct
DPVLTQSPSFLSLTVGQRVTINCQASQSISSYLAWYQQKPGQPPKLLIYRASTLASGVPSRFSGSGSGTEFTLTISSLQSDDFATYYCQCTDYGYNYLGAFGGGTRVEIK

SEQ ID NO: 37 - DNA, 363 bp, synthetic construct
  source 1..363: mol_type = other DNA
caggagcagctgaaggagtccgggggaggcctggtccagcctgggggatccctgacactcacctgcaaagcctctggattctccttcagtagcgtctactacatgtgctgggtccgccaggctccagggaaggggctggagtggatcggatgcagttatacgagtgatggtagcgcttactacgcgaactgggcgacaggccgattcaccatctccaaaacctcgtcgaccacggtgactctgcaaatgacgagtctgacagacgcggacacggccacctatttctgtgcgagagataggactgatagtcgtgattatttttttgacttgtggggcccaggcaccctggtcaccgtctcgagc

SEQ ID NO: 38 - protein, 121 aa, synthetic construct
QEQLKESGGGLVQPGGSLTLTCKASGFSFSSVYYMCWVRQAPGKGLEWIGCSYTSDGSAYYANWATGRFTISKTSSTTVTLQMTSLTDADTATYFCARDRTDSRDYFFDLWGPGTLVTVSS

SEQ ID NO: 39 - DNA, 333 bp, synthetic construct
  source 1..333: mol_type = other DNA
gcctatgatatgacccagactccagcctccgtggaggcagctgtgggaggcacagtcagcatcagttgtcagtccagtcagaatgtttatagtaactacttatcctggtatcagcagaaaccagggcagcctcccaagctcctgatctacagggcatccactctggcttctggggtctcatcgcgattcagaggcagtggatccgggacacagttcactctcaccatcagcggcgtggagtgtgccgatgctgccacttactattgtcaacagggttataaatatggtaatgttaataatcttttcggcggagggaccgaggtggaggtcaaa

SEQ ID NO: 40 - protein, 111 aa, synthetic construct
AYDMTQTPASVEAAVGGTVSISCQSSQNVYSNYLSWYQQKPGQPPKLLIYRASTLASGVSSRFRGSGSGTQFTLTISGVECADAATYYCQQGYKYGNVNNLFGGGTEVEVK

SEQ ID NO: 41 - DNA, 363 bp, synthetic construct
  source 1..363: mol_type = other DNA
gaggagcagctgctggagtccggcggcggcctggtgcagcccggcggctccctgaggctgtcctgcaccgcctccggcttcaccttcgagtccgtgtactacatgtgctgggtgaggcaggcccccggcaagggcctggagtggatcggctgctcctacacctccgacggctccgcctactacgccaactgggccaccggcaggttcaccatctccaagacctccatcaacaccgtgttcctgcagatgaggtccctgaggtccgaggacaccgccatctactactgcgccagggacaggaccgactccagggactacttcttcgacctgtggggccccggcaccctggtgaccgtgtcctcc

SEQ ID NO: 42 - protein, 121 aa, synthetic construct
EEQLLESGGGLVQPGGSLRLSCTASGFTFESVYYMCWVRQAPGKGLEWIGCSYTSDGSAYYANWATGRFTISKTSINTVFLQMRSLRSEDTAIYYCARDRTDSRDYFFDLWGPGTLVTVSS

SEQ ID NO: 43 - DNA, 333 bp, synthetic construct
  source 1..333: mol_type = other DNA
gcctacaggatgacccagtccccctcctccttctccgcctccaccggccagagggtgaccatcacctgccagtcctcccagaacgtgtactccaactacctgtcctggtaccagcagaagcccggcaagccccccaagctgctgatctacagggcctccaccctggcctccggcgtgccctccaggttcaggggctccggctccggcacccagttcaccctgaccatctcctgcgtgcagtgcgacgacttcgccacctactactgccagcagggctacaagtacggcaacgtgaacaacctgttcggcggcggcaccagggtggagatcaag

SEQ ID NO: 44 - protein, 111 aa, synthetic construct
AYRMTQSPSSFSASTGQRVTITCQSSQNVYSNYLSWYQQKPGKPPKLLIYRASTLASGVPSRFRGSGSGTQFTLTISCVQCDDFATYYCQQGYKYGNVNNLFGGGTRVEIK

SEQ ID NO: 45 - DNA, 363 bp, synthetic construct
  source 1..363: mol_type = other DNA
caggagcagctggtggagtccggcggcggcctggtgcagcccggcggctccctgaggctgtcctgcgccgcctccggcttcaccttctcctccgtgtactacatgtgctgggtgaggcaggcccccggcaagggcctggagtgggtgggctgctcctacacctccgacggctccgcctactacgccaactgggccaccggcaggttcaccatctccaagacctccaagaacaccgtgtacctgcagatgaactccctgagggccgaggacaccgccgtgtactactgcgccagggacaggaccgactccagggactacttcttcgacctgtggggccagggcaccctggtgaccgtgtcctcc

SEQ ID NO: 46 - protein, 121 aa, synthetic construct
QEQLVESGGGLVQPGGSLRLSCAASGFTFSSVYYMCWVRQAPGKGLEWVGCSYTSDGSAYYANWATGRFTISKTSKNTVYLQMNSLRAEDTAVYYCARDRTDSRDYFFDLWGQGTLVTVSS

SEQ ID NO: 47 - DNA, 333 bp, synthetic construct
  source 1..333: mol_type = other DNA
gcctacgtgatgacccagtccccctcctccctgtccgcctccgtgggcgacagggtgaccatctcctgccagtcctcccagaacgtgtactccaactacctgtcctggtaccagcagaagcccggccaggcccccaagctgctgatctacagggcctccaccctggcctccggcgtgccctccaggttctccggctccggctccggcaccgacttcaccctgaccatctcctccgtggagcccgaggacttcgccacctactactgccagcagggctacaagtacggcaacgtgaacaacctgttcggcggcggcaccaaggtggagatcaag

SEQ ID NO: 48 - protein, 111 aa, synthetic construct
AYVMTQSPSSLSASVGDRVTISCQSSQNVYSNYLSWYQQKPGQAPKLLIYRASTLASGVPSRFSGSGSGTDFTLTISSVEPEDFATYYCQQGYKYGNVNNLFGGGTKVEIK

SEQ ID NO: 49 - DNA, 363 bp, synthetic construct
  source 1..363: mol_type = other DNA
caggagcagctgctggagtccggcggcggcctggtgcagcccggcggctccctgaggctgtcctgcgccgcctccggcttcaccttctcctccgtgtactacatgtgctgggtgaggcaggcccccggcaagggcctggagtgggtgggctgctcctacacctccgacggctccgcctactacgccaactgggccaccggcaggttcaccatctccaagacctccaagaacaccgtgtacctgcagatgaactccctgagggccgaggacaccgccgtgtactactgcgccagggacaggaccgactccagggactacttcttcgacctgtggggccagggcaccctggtgaccgtgtcctcc

SEQ ID NO: 50 - protein, 121 aa, synthetic construct
QEQLLESGGGLVQPGGSLRLSCAASGFTFSSVYYMCWVRQAPGKGLEWVGCSYTSDGSAYYANWATGRFTISKTSKNTVYLQMNSLRAEDTAVYYCARDRTDSRDYFFDLWGQGTLVTVSS

SEQ ID NO: 51 - DNA, 333 bp, synthetic construct
  source 1..333: mol_type = other DNA
gcctacaggatgacccagtccccctcctccgtgtccgcctccgtgggcgacagggtgaccatctcctgccagtcctcccagaacgtgtactccaactacctgtcctggtaccagcagaagcccggccaggcccccaagctgctgatctacagggcctccaccctggcctccggcgtgccctccaggttctccggctccggctccggcaccgacttcaccctgaccatctccggcgtgcagtccgaggacttcgccacctactactgccagcagggctacaagtacggcaacgtgaacaacctgttcggcggcggcaccaaggtggagatcaag

SEQ ID NO: 52 - protein, 111 aa, synthetic construct
AYRMTQSPSSVSASVGDRVTISCQSSQNVYSNYLSWYQQKPGQAPKLLIYRASTLASGVPSRFSGSGSGTDFTLTISGVQSEDFATYYCQQGYKYGNVNNLFGGGTKVEIK

SEQ ID NO: 53 - DNA, 351 bp, synthetic construct
  source 1..351: mol_type = other DNA
cagtcggtggaggagtccgggggtcgcctggtcacgcctgggacacccctgacactcacctgcacggtctctggattctccctcagtacctattcaatgggctgggtccgccaggctccagggaaggggctggaatggatcggagtcatttactatggtgagcccacatactgcgcggcctgggcgaaaggccgattcaccatctccaaaacctcgtcgaccacggtggatctgaaaatcaccagtccgacaaccgaggacacggccacctatttctgtgccagatcaggtgatcgtaatggctactactttaacatctggggcccaggcaccctggtcaccgtctcgagc

SEQ ID NO: 54 - protein, 117 aa, synthetic construct
QSVEESGGRLVTPGTPLTLTCTVSGFSLSTYSMGWVRQAPGKGLEWIGVIYYGEPTYCAAWAKGRFTISKTSSTTVDLKITSPTTEDTATYFCARSGDRNGYYFNIWGPGTLVTVSS

SEQ ID NO: 55 - DNA, 333 bp, synthetic construct
  source 1..333: mol_type = other DNA
gcctatgatatgacccagactccagcctccgtgtctgcagctgtgggaggcacagtcaccatcaagtgccaggccagtcagagcattagtagctacttagcctggtatcagcagaaaccagggcagcctcccaagctcctgatctattatgcatccactctggcatctggggtcccatcgcggttcaaaggcagtggatctgggacagagttcactctcaccatcagcggcgtgcagtgtgccgatgcagccacttactactgtcaacaggttgctgttattggtaatgttggggataatactttcggcggagggaccgaggtggaggtcaaa

SEQ ID NO: 56 - protein, 111 aa, synthetic construct
AYDMTQTPASVSAAVGGTVTIKCQASQSISSYLAWYQQKPGQPPKLLIYYASTLASGVPSRFKGSGSGTEFTLTISGVQCADAATYYCQQVAVIGNVGDNTFGGGTEVEVK

SEQ ID NO: 57 - protein, 5 aa, synthetic construct
GFAMG

SEQ ID NO: 58 - protein, 16 aa, synthetic construct
FIDSGGGAFYATWARG

SEQ ID NO: 59 - protein, 12 aa, synthetic construct
HGGNTYYYAMDP

SEQ ID NO: 60 - protein, 11 aa, synthetic construct
QASQSISNYLA

SEQ ID NO: 61 - protein, 7 aa, synthetic construct
SASTLAS

SEQ ID NO: 62 - protein, 12 aa, synthetic construct
QQVYSVTNIDNA

SEQ ID NO: 63 - protein, 5 aa, synthetic construct
TYAMS

SEQ ID NO: 64 - protein, 16 aa, synthetic construct
IIDAVGRTYYASWAKS

SEQ ID NO: 65 - protein, 4 aa, synthetic construct
WWDL

SEQ ID NO: 66 - protein, 13 aa, synthetic construct
QSSQSVYDSNDLT

SEQ ID NO: 67 - protein, 11 aa, synthetic construct
LASTLASGVPS

SEQ ID NO: 68 - protein, 10 aa, synthetic construct
AAHYNSDIYT

SEQ ID NO: 69 - protein, 5 aa, synthetic construct
SNGMS

SEQ ID NO: 70 - protein, 16 aa, synthetic construct
FIDTDGSAYSASWAKG

SEQ ID NO: 71 - protein, 13 aa, synthetic construct
AYVSGSTGYNFNI

SEQ ID NO: 72 - protein, 11 aa, synthetic construct
QASQSISSYLA

SEQ ID NO: 73 - protein, 7 aa, synthetic construct
KASTLAS

SEQ ID NO: 74 - protein, 12 aa, synthetic construct
QQGYTIRNIDNA

SEQ ID NO: 75 - protein, 5 aa, synthetic construct
SYSMG

SEQ ID NO: 76 - protein, 16 aa, synthetic construct
YLNTGGSTYYASWVNG

SEQ ID NO: 77 - protein, 12 aa, synthetic construct
AGGVSSNMGFNI

SEQ ID NO: 78 - protein, 11 aa, synthetic construct
QASQSISSYLA

SEQ ID NO: 79 - protein, 7 aa, synthetic construct
RASTLAS

SEQ ID NO: 80 - protein, 12 aa, synthetic construct
QCTDYGYNYLGA

SEQ ID NO: 81 - protein, 5 aa, synthetic construct
TYSMG

SEQ ID NO: 82 - protein, 16 aa, synthetic construct
VIYYGEPTYCAAWAKG

SEQ ID NO: 83 - protein, 11 aa, synthetic construct
SGDRNGYYFNI

SEQ ID NO: 84 - protein, 11 aa, synthetic construct
QASQSISSYLA

SEQ ID NO: 85 - protein, 7 aa, synthetic construct
YASTLAS

SEQ ID NO: 86 - protein, 13 aa, synthetic construct
QQVAVIGNVGDNT

SEQ ID NO: 87 - DNA, 786 bp, synthetic construct
  source 1..786: mol_type = other DNA
atggccgtgaccgcctgccagggcctgggcttcgtggtgagcctgatcggcatcgccggcatcatcgccgccacctgcatggaccagtggagcacccaggacctgtacaacaaccccgtgaccgccgtgttcaactaccagggcctgtggaggagctgcgtgagggagagcagcggcttcaccgagtgcaggggctacttcaccctgctgggcctgcccgccatgctgcaggccgtgagggccctgatgatcgtgggcatcgtgctgggcgccatcggcctgctggtgagcatcttcgccctgaagtgcatcaggatcggcagcatggaggacagcgccaaggccaacatgaccctgaccagcggcatcatgttcatcgtgagcggcctgtgcgccatcgccggcgtgagcgtgttcgccaacatgctggtgaccaacttctggatgagcaccgccaacatgtacaccggcatgggcggcatggtgcagaccgtgcagaccaggtacaccttcggcgccgccctgttcgtgggctgggtggccggcggcctgaccctgatcggcggcgtgatgatgtgcatcgcctgcaggggcctggcccccgaggagaccaactacaaggccgtgagctaccacgccagcggccacagcgtggcctacaagcccggcggcttcaaggccagcaccggcttcggcagcaacaccaagaacaagaagatctacgacggcggcgccaggaccgaggacgaggtgcagagctaccccagcaagcacgactacgtgtga

SEQ ID NO: 88 - DNA, 786 bp, synthetic construct
  source 1..786: mol_type = other DNA
atgtccaccaccacatgccaagtggtggcgttcctcctgtccatcctggggctggccggctgcatcgcggccaccgggatggacatgtggagcacccaggacctgtacgacaaccccgtcacctccgtgttccagtacgaagggctctggaggagctgcgtgaggcagagttcaggcttcaccgaatgcaggccctatttcaccatcctgggacttccagccatgctgcaggcagtgcgagccctgatgatcgtaggcatcgtcctgggtgccattggcctcctggtatccatctttgccctgaaatgcatccgcattggcagcatggaggactctgccaaagccaacatgacactgacctccgggatcatgttcattgtctcaggtctttgtgcaattgctggagtgtctgtgtttgccaacatgctggtgactaacttctggatgtccacagctaacatgtacaccggcatgggtgggatggtgcagactgttcagaccaggtacacatttggtgcggctctgttcgtgggctgggtcgctggaggcctcacactaattgggggtgtgatgatgtgcatcgcctgccggggcctggcaccagaagaaaccaactacaaagccgtttcttatcatgcctcaggccacagtgttgcctacaagcctggaggcttcaaggccagcactggctttgggtccaacaccaaaaacaagaagatatacgatggaggtgcccgcacagaggacgaggtacaatcttatccttccaagcacgactatgtgtaa

(19) United States
(12) Patent Application Publication
(10) Pub. No.: US 2026/0317420 A1
(43) Pub. Date: Oct. 15, 2026

(54) ANTI-CLAUDIN18.2 ANTIBODY AND METHOD OF MAKING AND USING THEREOF

(71) Applicant: SYSTIMMUNE, INC., Redmond, WA (US)

(72) Inventors: Kelcey D. Dinkel, Redmond, WA (US); Dennis R. Goulet, Redmond, WA (US); Ana Dina, Seattle, WA (US); Yi Zhu, Redmond, WA (US)

(73) Assignee: SYSTIMMUNE, INC., Redmond, WA (US)

(21) Appl. No.: 19/484,159

(22) PCT Filed: May 21, 2024
(86) PCT No.: PCT/US2024/030371

§ 371 Date: Nov. 13, 2025

Related U.S. Application Data
(60) Provisional application No. 63/468,198, filed on May 22, 2023.

(51) Int. Cl. C07K 16/28; A61K 45/06; A61K 47/68

(52) U.S. Cl. CPC C07K 16/28; A61K 45/06; A61K 47/68031; A61K 47/68033; A61K 47/6809; A61K 47/6849; A61K 47/6889; C07K 2317/24; C07K 2317/31; C07K 2317/565; C07K 2317/567; C07K 2317/732; C07K 2317/734; C07K 2317/77; C07K 2317/92; C07K 2317/94

(57) ABSTRACT

An isolated monoclonal antibody (mAb) or antigen-binding fragment thereof having a binding specificity to CLDN18, wherein the isolated mAb or antigen-binding fragment thereof selectively binds to CLDN18.2 with measurable affinity and avidity, but not CLDN18.1.

### CROSS-REFERENCE TO RELATED APPLICATIONS

This application claims the benefit of the filing date of U.S. Provisional Application Ser. No. 63/468,198 filed May 22, 2023, under 35 U.S.C. 119(e), the entire disclosures of which are incorporated by reference herein.

### TECHNICAL FIELD

The present disclosure generally relates to the technical field of antibody cancer therapeutics, and more particularly relates to anti-Claudin18.2 antibodies.

### BACKGROUND

Gastric and pancreatic cancers are among the most common malignancies worldwide. Gastric cancer is a major cause of death in many parts of Asia, South and Central America, and Eastern Europe. In 2020, it was ranked the 5^{th} most common cancer type with more than 1 million new cases reported¹. In the US, it was estimated that 26,380 cases would be diagnosed in 2022 and that death would occur in 11,090 of those cases. Between 2011 and 2017, the combined overall 5-year survival rate was 32%². Pancreatic cancer is the 12th most common cancer worldwide, with more than 495,000 cases reported in 2020³. In the US, pancreatic cancer is more common than gastric cancer, accounting for 3% of cancers and 7% of cancer deaths⁴. Due to the advanced stage of disease following diagnosis, the prognosis is poor with a combined 5-year survival rate of only 11%². While targeted therapeutics are improving, these malignancies represent an area of clearly unmet medical need.

The overexpression of Claudin18.2 (aka, CLDN18.2) has been linked to several human malignancies, including gastric and pancreatic cancers. CLDN18.2 is a selective marker for gastric mucosal epithelial cells and, during normal expression, is buried within the tight junctions of healthy tissues⁵. However, during carcinogenesis, the disruption of the epithelial cell barrier allows for the exposure of CLDN18.2 epitopes that can be accessed by targeted therapeutics⁵⁻⁷. This distinction makes CLDN18.2 a unique, gastric cancer-specific target for immune therapies. In addition to gastric cancer, the overexpression of CLDN18.2 has been detected in several primary cancers, including breast, colon, esophageal, biliary tract, and pancreatic cancers⁶. While CLDN18.2 is not detected in normal pancreatic tissues, in cases of primary pancreatic ductal adenocarcinoma, 59.2% of cases had cells with CLDN18.2 positive expression⁵. Several antibodies targeting CLDN18.2 for cancer immunotherapy are in development; however, none have been approved for clinical use⁵. Therefore, a need remains for the development of highly specific anti-CLDN18.2 antibodies for treating malignancies such as gastric and pancreatic cancers.

### SUMMARY

The disclosure generally relates to the technical field of antibody therapeutic agents, and more particularly relates to antibodies against specific epitopes of Claudin18, including CLDN18.1 and/or CLDN18.2.

In one aspect, the disclosure provides isolated monoclonal antibodies (mAbs) or antigen-binding fragments thereof having a binding specificity to CLDN18.1, CLDN18.2, or a combination thereof. In one embodiment, the mAbs or antigen-binding fragment thereof have a binding affinity to CLDN18.1. In one embodiment, the mAbs or antigen-binding fragment thereof have a binding affinity to CLDN18.2. In one embodiment, the mAbs or antigen-binding fragment thereof have a binding affinity to CLDN18.1 and CLDN 18.2.

In one embodiment, the isolated mAbs or antigen-binding fragment thereof comprise an amino acid sequence having at least 70%, 80%, 85%, 90%, 95%, 98%, 99%, or 100% identity with the SEQ ID NO: 2, 4, 6, 8, 10, or 12. In one embodiment, the isolated mAbs or antigen-binding fragment thereof comprise an amino acid sequence having at least 70%, 80%, 85%, 90%, 95%, 98%, 99%, or 100% identity with the SEQ ID NO: 14 or 16. In one embodiment, the isolated mAbs or antigen-binding fragment thereof comprise an amino acid sequence having at least 70%, 80%, 85%, 90%, 95%, 98%, 99%, or 100% identity with the SEQ ID NO:18, 20, 22, 24, 26, or 28. In one embodiment, the isolated mAbs or antigen-binding fragment thereof comprise an amino acid sequence having at least 70%, 80%, 85%, 90%, 95%, 98%, 99%, or 100% identity with the SEQ ID NO: 30, 32, 34, or 36. In one embodiment, the isolated mAbs or antigen-binding fragment thereof comprise an amino acid sequence having at least 70%, 80%, 85%, 90%, 95%, 98%, 99%, or 100% identity with the SEQ ID NO: 38, 40, 42, 44, 46, 48, 50, or 52. In one embodiment, the isolated mAbs or antigen-binding fragment thereof comprise an amino acid sequence having at least 70%, 80%, 85%, 90%, 95%, 98%, 99%, or 100% identity with the SEQ ID NO: 54 or 56.

The expression of CLDN18.2 is associated with several human malignancies, including gastric and pancreatic cancers. The disclosure further discloses the development of anti-CLDN18.2 antibodies for treating cancer.

In another aspect, the disclosure provides an isolated mAb or antigen-binding fragment thereof having a binding specificity to CLDN18, comprising an amino acid sequence having at least 70%, 80%, 85%, 90%, 95%, 98%, 99%, or 100% identity with the SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, or 56.

In one embodiment, the isolated mAb or antigen-binding fragment thereof selectively binds to CLDN18.2 but not CLDN18.1. In one embodiment, isolated mAb or antigen-binding fragment thereof has a binding affinity to CLDN18.2 having a KD value of not more than 5, 3, 2, 1.5, 1.45, 1.4, 1, or 0.8 nM. In one embodiment, isolated mAb or antigen-binding fragment thereof has a binding avidity to CLDN18.2 having a KD value of not more than 0.5, 0.4, 0.3, 0.25, 0.24, 0.23, 0.22, 0.21, 0.2, or 0.18 nM. In one embodiment, the ratio of binding of the isolated mAb or antigen-binding fragment thereof to CLDN18.2 to CLDN18.1 is at least 4, 5, 8, 10, 15, 20, 30, or 40. In one embodiment, the ratio of binding of the isolated mAb or antigen-binding fragment thereof to CLDN18.2 to CLDN18.1 is from about 4 to about 10, from about 3 to about 12, from about 10 to about 25, or from about 12 to about 22.

In one embodiment, the isolated mAb or antigen-binding fragment further includes a human framework region.

In one embodiment, the isolated mAb may be a humanized antibody, a chimeric antibody, or a recombinant antibody. In one embodiment, the isolated mAb is an IgG. In one embodiment, the isolated mAb may be a bispecific antibody, tri-specific antibody, or multi-specific antibody.

In one embodiment, the antigen-binding fragment may include a Fv, a Fab, a F(ab′)2, a scFv or a scFv2 fragment.

In one embodiment, the disclosure provides an IgG1 heavy chain for the isolated mAb or antigen-binding fragment thereof, comprising an amino acid sequence having at least 70%, 80%, 85%, 90%, 95%, 98%, 99%, or 100% identity with the SEQ ID NO: 2, 6, 10, 14, 18, 22, 26, 30, 34, 38, 42, 46, 50, or 54.

In one embodiment, the disclosure provides a kappa light chain for the isolated mAb or antigen-binding fragment as disclosed herein, comprising an amino acid sequence having at least 70%, 80%, 85%, 90%, 95%, 98%, 99%, or 100% identity the SEQ ID NO: 4, 8, 12, 16, 20, 24, 28, 32, 36, 40, 44, 48, 52, or 56.

In one embodiment, the disclosure provides the variable light chain for the isolated mAb or antigen-binding fragment as disclosed herein, comprising an amino acid sequence having at least 70%, 80%, 85%, 90%, 95%, 98%, 99%, or 100% identity with the SEQ ID NO: 4, 8, 12, 16, 20, 24, 28, 32, 36, 40, 44, 48, 52, or 56.

In one embodiment, the disclosure provides the variable heavy chain for the isolated mAb or antigen-binding fragment as disclosed herein, comprising an amino acid sequence having at least 70%, 80%, 85%, 90%, 95%, 98%, 99%, or 100% identity with the SEQ ID NO: 2, 6, 10, 14, 18, 22, 26, 30, 34, 38, 42, 46, 50, or 54.

In one embodiment, the disclosure provides an isolated mAb or antigen-binding fragment thereof having a binding specificity to CLDN18. In one embodiment, the isolated mAb or antigen-binding fragment thereof comprises a variable heavy chain and a variable light chain having complementarity determining region (CDR)H1, CDRH2, CDRH3, CDRL1, CDRL2, and CDRL3 of SEQ ID NO: 57, 58, 59, 60, 61, and 62; 63, 64, 65, 66, 67, and 68; 69, 70, 71, 72, 73, and 74; 75, 76, 77, 78, 79, and 80; or 81, 82, 83, 84, 85, and 86.

In a further aspect, the disclosure provides an isolated nucleic acid encoding the isolated mAb or antigen-binding fragment, the IgG1 heavy Chain, the kappa light chain, the variable light chain, or the variable heavy chain, as disclosed herein. In one embodiment, the isolated nucleic acid has at least 70%, 80%, 85%, 90%, 95%, 98%, 99%, or 100% identity with the SEQ ID. NO:1, 3, 5, 7, 9, 11, 13, 15, 17, 19, 21, 23, 25, 27, 29, 31, 33, 35, 37, 39, 41, 43, 45, 47, 49, 51, 53, or 55.

In a further aspect, the disclosure provides an expression vector comprising the isolated nucleic acid as disclosed herein, wherein the vector is expressible in a cell.

In a further aspect, the disclosure provides a host cell comprising the nucleic acid as disclosed herein, wherein the host cell is a prokaryotic cell or a eukaryotic cell.

In a further aspect, the disclosure provides a method of producing an antibody comprising culturing the host cell as disclosed herein, so that the antibody is produced.

In a further aspect, the disclosure provides an immuno-conjugate, comprising the isolated mAb or antigen-binding fragment thereof conjugated to a drug unit through a linker. In one embodiment, the linker comprises a covalent bond selected from an ester bond, an ether bond, an amine bond, an amide bond, a disulfide bond, an imide bond, a sulfone bond, a phosphate bond, a phosphorus ester bond, a peptide bond, a hydrazone bond or a combination thereof.

In one embodiment, the drug unit comprises a cytotoxic agent, an immune regulatory reagent, or a combination thereof. In one embodiment, cytotoxic agent is selected from a growth inhibitory agent or a chemotherapeutic agent from a class of tubulin binders, DNA intercalators, DNA alkylators, enzyme inhibitors, immune modulators, antimetabolite agents, radioactive isotopes or a combination thereof. In one embodiment, the cytotoxic agent is selected from a calicheamicin, ozogamicin, monomethyl auristatin E, emtansine a derivative or a combination thereof. In one embodiment, the immune regulatory reagents activate or suppress immune cells, T cell, NK cell, B cell, macrophage, or dendritic cell.

In a further aspect, the disclosure provides a pharmaceutical composition, comprising the isolated mAb or antigen-binding fragment thereof or the immuno-conjugate, and a pharmaceutically acceptable carrier.

In one embodiment, the pharmaceutical composition further comprises a radioisotope, a radionuclide, a toxin, a therapeutic agent, a chemotherapeutic agent, or a combination thereof.

In one embodiment, the therapeutic agent comprises an anti-estrogen agent, a receptor tyrosine kinase inhibitor, a kinase inhibitor, a cell cycle inhibitor, a DNA, RNA or protein synthesis inhibitor, a RAS inhibitor, or a combination thereof.

In a further aspect, the disclosure provides a method of treating a subject with cancer. In one embodiment, the method comprises administering to the subject an effective amount of the isolated mAb or antigen-binding fragment thereof. In one embodiment, the method comprises administering to the subject an effective amount of the immuno-conjugate as disclosed herein. In one embodiment, the method comprises administering to the subject an effective amount of the pharmaceutical composition as disclosed herein.

In one embodiment, the cancer comprises cells expressing Claudin 18.2. In one embodiment, the cancer comprises colorectal cancer, pancreatic cancer, esophageal cancer, nasopharyngeal cancer, anal cancer, rectal cancer, gastric cancer, or bladder cancer.

In one embodiment, the method of treating cancer further comprises co-administering an effective amount of a therapeutic agent. In one embodiment, the therapeutic agent comprises an antibody, a chemotherapy agent, an enzyme, anti-estrogen agent, a receptor tyrosine kinase inhibitor, a kinase inhibitor, a cell cycle inhibitor, a DNA, RNA or protein synthesis inhibitor, a RAS inhibitor or a combination thereof.

In one embodiment, the subject is a human.

In a further aspect, the disclosure provides a solution comprising an effective concentration of the isolated mAb or an antigen-binding fragment thereof, wherein the solution is blood plasma in a subject. In one embodiment, the disclosure provides a solution comprising an effective concentration of the immune-conjugate as disclosed herein. In one embodiment, the solution is blood plasma in the subject.

### BRIEF DESCRIPTION OF THE DRAWINGS

The foregoing and other features of this disclosure may become more fully apparent from the following description and appended claims, taken in conjunction with the accompanying drawings. Understanding that these drawings depict only several embodiments arranged in accordance with the disclosure and are, therefore, not to be considered limiting of its scope, the disclosure may be described with additional specificity and detail through use of the accompanying drawings, in which:

FIG. 1 shows the alignment of extracellular claudin domains (ECD1 and ECD2) in the full-length amino acid sequences encoding human and cynomolgus monkey Claudin18.1 and Claudin18.2;

FIG. 2 shows the detection of anti-Claudin18.2 antibodies in B cell culture screen by direct sandwich ELISA;

FIG. 3 shows the detection of anti-Claudin18.2 antibodies in B cell culture screen by flow cytometry using 293 HEK transfected with either a Claudin18.2 or a Claudin18.1 expression vector;

FIG. 4 shows the characterization of chimeric anti-Claudin18.2 antibodies by flow cytometry using ExpiCHO cells transfected with either a Claudin18.2 or a Claudin18.1 expression vector;

FIG. 5 shows the characterization of binding affinity of chimeric anti-Claudin18.2 antibodies, 5208H10 (5A) and 5103F3 (5B), by Bio-Layer Interferometry followed by fits and calculations using manufacturer-provided software followed by fits and calculations using manufacturer-provided software;

FIG. 6 shows the characterization of binding avidity of chimeric anti-Claudin18.2 antibodies by Bio-Layer Interferometry followed by fits and calculations using manufacturer-provided software;

FIG. 7 shows the characterization of CLDN18.2 specificity of humanized anti-Claudin18.2 antibody by flow cytometry and ExpiCHO cells transiently expressing BCMA-Claudin18 fusion proteins;

FIG. 8 shows the characterization of CLDN18.2 specificity of humanized anti-Claudin18.2 antibody by direct ELISA using recombinant protein VLPs;

FIG. 9 shows the characterization of epitope binning of anti-Claudin18.2 antibodies by Bio-Layer Interferometry (OCTET);

FIG. 10 shows the lysis of CLDN18.2-exppressing CHO cells (CHO-Claudin18.2) in response to increasing doses of humanized anti-Claudin18.2 antibodies in Complement-Dependent Cytotoxicity (CDC) assay;

FIG. 11 shows the lysis of Claudin18.2-exppressing CHO cells (CHO-Claudin18.2) in the presence of donor NK cells as effector cells (5:1 to target cells) and increasing doses of humanized anti-Claudin18.2 antibodies in Antibody-Dependent Cell-mediated Cytotoxicity (ADCC) assay;

FIG. 12 shows the characterization of cross-specific interactions among anti-Claudin 18.2 antibodies of human, rat, and murine origins by flow cytometry;

FIG. 13 shows the characterization of lysosomal trafficking of anti-Claudin18.2 antibodies at 5 nM and 10 nM concentrations by using time-series live-cell fluorescence microscopy; and

FIG. 14 shows the sequence alignment between 5103F3 BSM and comparator antibodies (14A); the % identities of all CDRs and framework only (14B, upper and lower panels, respectively), and the % identities of the entire variable region (14C).

### DETAILED DESCRIPTION

The disclosure discloses the generation, characterization, and application of anti-CLDN18.2 antibodies for treating malignancies such as gastrointestinal tumors. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

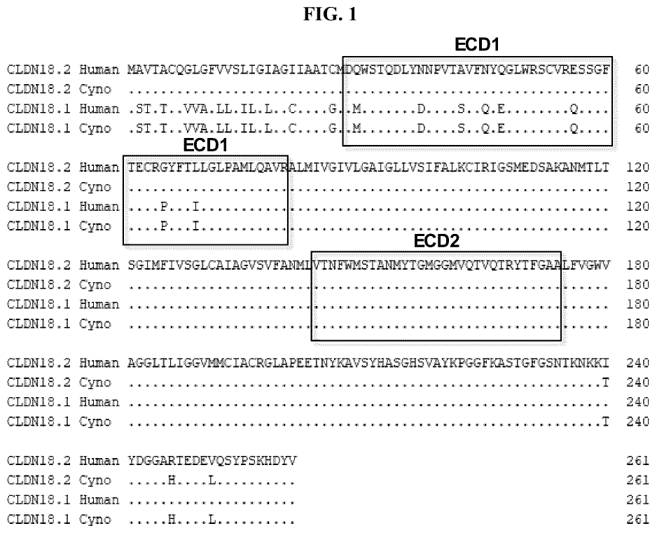

Claudins (aka, CLDNs) are a family of proteins that have direct roles in the formation of tight junctions. Tight junctions form a paracellular barrier that controls the flow of molecules between cells. CLDNs are critical for the formation of these barriers and help to maintain the polarity of the epithelial cell layers⁸. CLDNs are tetraspanin proteins composed of four transmembrane domains: N- and C-terminal ends within the cytoplasm, and two extracellular domain loops⁸. CLDNs are expressed in normal tissues; however, altered function and expression have been linked to the formation of cancer^{5-6,8}. Of particular interest is Claudin18.2 (CLDN18.2), which has been linked to several human malignancies, including gastric and pancreatic cancers⁵⁻⁶. The human CLDN18 gene contains 6 exons and 5 introns. Alternative splicing of exons 1a and 1b forms two isoforms, CLDN18.1 and CLDN18.2⁹. These splice variants are restricted to different tissues, with CLDN18.1 predominantly expressed in normal lung tissue and CLDN18.2 expressed in gastric tissue⁵⁻⁶. The sequence of CLDN18.1 is almost identical to CLDN18.2 with only 8 amino acid differences within extracellular loop D1 (ECDs in FIG. 1). It is this high sequence similarity that makes the discovery of CLDN18.2-specific antibodies significantly more challenging.

Several antibodies targeting CLDN18.2 for cancer immunotherapy are in development, including monospecific monoclonal antibodies (mAbs), bispecific antibodies (BsAbs), chimeric antigen receptor T cells (CAR-T), and antibody-drug conjugates (ADC). Zolbetuximab (Astellas Pharma) is a mouse chimeric mAb that specifically binds CLDN18.2 and has shown safety and efficacy for patients with HER2-positive advanced gastric cancer⁵. TST001 (Mabspace Bio-Sciences) is a humanized mAb that has shown comparable activity to Zolbetuximab⁵. While the majority of antibodies under development are mAbs, BsAbs and ADCs have also proven to be valid methods of treatment for both gastric and pancreatic cancers, including AMG-910 (Amgen, bispecific T-cell engager) and CMG901 (Keymed Biosciences, ADC)⁵. However, no CLDN18.2-targeting antibodies have been approved for clinical application.

The present disclosure provides, among others, isolated antibodies, methods of making such antibodies, monoclonal and/or recombinant monospecific antibodies, pharmaceutical compositions containing the antibodies, monoclonal and/or recombinant monospecific antibodies, the methods for making the antibodies and compositions, and the methods for treating cancer using the antibodies and compositions disclosed herein. Specifically, the present disclosure provides isolated monospecific monoclonal antibodies (mAb) or antigen-binding fragments thereof having a binding specificity to human Claudin18.2, wherein the isolated mAb or antigen-binding fragments comprise an amino acid sequence having an identity with a sequence selected from SEQ ID NO. 2, 4, 7, 8, 10, 12, 14, 16, 18, 20, 22, 24, 26, 28, 30, 32, 34, 36, 38, 40, 42, 44, 46, 48, 50, 52, 54, or 56.

The terms “a”, “an” and “the” as used herein are defined to mean “one or more” and include the plural unless the context is inappropriate.

The terms “polypeptide”, “peptide”, and “protein”, as used herein, are interchangeable and are defined to mean a biomolecule composed of amino acids linked by a peptide bond.

The term “antigen” refers to an entity or fragment thereof which can induce an immune response in an organism, particularly an animal, more particularly a mammal including a human. The term includes immunogens and regions thereof responsible for antigenicity or antigenic determinants.

The terms “antigen- or epitope-binding portion or fragment”, “variable region”, “variable region sequence”, or “binding domain” refer to fragments of an antibody that are capable of binding to an antigen (such as Claudin18.2 in this application). These fragments may be capable of the antigen-binding function and additional functions of the intact antibody. Examples of binding fragments include, but are not limited to, a single-chain Fv fragment (scFv) consisting of the variable light chain (VL) and variable heavy chain (VH) domains of a single arm of an antibody connected in a single polypeptide chain by a synthetic linker, or a Fab fragment which is a monovalent fragment consisting of the VL, constant light (CL), VH and constant heavy 1 (CH1) domains. Antibody fragments can be even smaller sub-fragments and can consist of domains as small as a single CDR domain, in particular, the CDR3 regions from either the VL and/or VH domains (for example see Beiboer et al., J. Mol. Biol. 296:833-49 (2000)). Antibody fragments are produced using conventional methods known to those skilled in the art. The antibody fragments can be screened for utility using the same techniques employed with intact antibodies.

The “antigen- or epitope-binding portion or fragment”, “variable region”, “variable region sequence”, or “binding domain” may be derived from an antibody of the present disclosure by a number of art-known techniques. For example, purified monoclonal antibodies can be cleaved with an enzyme, such as pepsin, and subjected to HPLC gel filtration. Papain digestion of antibodies produces two identical antigen binding fragments, called “Fab” fragments, each with a single antigen binding site, and a residual “Fc” fragment, whose name reflects its ability to crystallize readily. Pepsin treatment yields an F(ab′)₂ fragment that has two antigen combining sites and is still capable of cross-linking antigen. The appropriate fraction containing Fab fragments can then be collected and concentrated by membrane filtration and the like. For further description of general techniques for the isolation of active fragments of antibodies, see for example, Khaw, B. A. et al. J. Nucl. Med. 23:1011-1019 (1982); Rousseaux et al. Methods Enzymology, 121:663-69, Academic Press, 1986.

The term “antibody” is used in the broadest sense and specifically covers single monoclonal antibodies and/or recombinant antibodies (including agonist and antagonist antibodies), antibody compositions with polyepitope specificity, as well as antibody fragments (e.g., Fab, F(ab′)₂, and Fv), so long as they exhibit the desired biological activity. In some embodiments, the antibody may be monoclonal, polyclonal, chimeric, single chain, multi-specific or multi-effective, human and humanized antibodies, and/or active fragments thereof. Examples of active fragments of molecules that bind to known antigens include Fab, F(ab′)₂, scFv and Fv fragments, including the products of a Fab immunoglobulin expression library and epitope-binding fragments of any of the antibodies and fragments mentioned above.

The term “Fv” refers to the minimum antibody fragment which contains a complete antigen recognition and binding site. This region consists of a dimer of one heavy and one light chain variable domain in tight, non-covalent association. It is in this configuration that the three CDRs of each variable domain interact to define an antigen binding site on the surface of the VH-VL dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

In some embodiments, antibody may include immunoglobulin molecules and immunologically active portions of immunoglobulin molecules, i.e., molecules that contain a binding site and that can immunospecifically bind to an antigen. A typical antibody refers to heterotetrameric protein comprising typically of two heavy (H) chains and two light (L) chains. Each heavy chain is comprised of a heavy chain variable domain (abbreviated as VH) and three heavy chain constant domains (abbreviated as CH1, CH2, and CH3). Each light chain is comprised of a light chain variable domain (abbreviated as VL) and a light chain constant domain (abbreviated as CL). The light chains of antibodies (immunoglobulins) from any vertebrate species can be assigned to one of two clearly distinct types, called kappa and lambda, based on the amino acid sequences of their constant domains. The VH and VL regions can be further subdivided into domains of hypervariable complementarity determining regions (CDRs), and more conserved regions called framework regions (FR). Each variable domain (either VH or VL) is typically composed of three CDRs and four FRs, arranged in the following order: FR1, CDR1, FR2, CDR2, FR3, CDR3, FR4 from amino-terminus to carboxy-terminus. Within the variable regions of the light and heavy chains there are binding regions that interacts with the antigen.

Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG1, IgG2, IgG3, and IgG4; IgA1 and IgA2. The heavy chain constant domains that correspond to the different classes of immunoglobulins are called alpha, delta, epsilon, gamma, and mu, respectively. The subunit structures and three-dimensional configurations of different classes of immunoglobulins are well known.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies and is not to be construed as requiring production of the antibody by any particular method. For example, the monoclonal antibodies to be used in accordance with the present disclosure may be made by the hybridoma method first described by Kohler & Milstein, Nature, 256:495 (1975), or may be made by recombinant DNA methods (see, e.g., U.S. Pat. No. 4,816,567). “Recombinant” means the antibodies are generated using recombinant nucleic acid techniques in exogeneous host cells.

Monoclonal antibodies can be produced using various methods, including without limitation, mouse hybridoma, phage display, recombinant DNA, molecular cloning of antibodies directly from primary B cells, and antibody discovery methods (see Siegel. Transfus. Clin. Biol. 2002; Tiller. New Biotechnol. 2011; Seeber et al. PLOS One. 2014). Monoclonal antibodies may include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 [1984]).

The term “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity. Methods to obtain “humanized antibodies” are well known to those skilled in the art. [see, e.g., Queen et al., Proc. Natl Acad Sci USA, 86:10029-10032 (1989), Hodgson et al., Bio/Technology, 9:421 (1991)).

The terms “antigen- or epitope-binding portion or fragment”, “variable domain”, “variable region”, “variable region sequence”, or “binding domain” refer to fragments of an antibody that are capable of binding to an antigen (such as EGFR and HER3 in this application). The antigen-binding fragment (Fab) is a region (Fab region) on an antibody that binds to antigens. These fragments may be capable of the antigen-binding function and additional functions of the intact antibody. Examples of binding fragments include, but are not limited to, a single-chain Fv fragment (scFv) consisting of the variable light chain (VL) and variable heavy chain (VH) domains of a single arm of an antibody connected in a single polypeptide chain by a synthetic linker, or a Fab fragment which is a monovalent fragment consisting of the VL, constant light (CL), VH and constant heavy 1 (CH1) domain.

The terms “antigen- or epitope-binding portion or fragment”, “variable region”, “variable region sequence”, or “binding domain” may be derived from an antibody of the disclosure by several art-known techniques. For example, purified monoclonal antibodies can be cleaved with an enzyme, such as pepsin, and subjected to HPLC gel filtration. Papain digestion of antibodies produces two identical antigen binding fragments, called “Fab” fragments, each with a single antigen binding site, and a residual “Fc” fragment, whose name reflects its ability to crystallize readily. Pepsin treatment yields an F(ab′)2 fragment that has two antigen combining sites and is still capable of cross-linking antigen. The appropriate fraction containing Fab fragments can then be collected and concentrated by membrane filtration and the like. For further description of general techniques for the isolation of active fragments of antibodies (see for example, Khaw, B. A. et al. J. Nucl. Med. 23:1011-1019 (1982); Rousseaux et al. Methods Enzymology, 121:663-69, Academic Press, 1986).

The term “Octet” refers Bio-Layer Interferometry (BLI), a label-free method for measuring bi-molecular interactions. The Dip and Read technology eliminate the need of microfluidics and means there is no need to purify antibodies to get valuable antigen binding and specificity information.

The present disclosure may be understood more readily by reference to the following detailed description of specific embodiments and examples included herein. Although the present disclosure has been described with reference to specific details of certain embodiments thereof, it is not intended that such details should be regarded as limitations upon the scope of the disclosure.

### EXAMPLES

### Example 1. Generation of Rabbit Antibodies to Claudin18

### Immunizations:

CLDN18.2-specific antibodies were obtained by immunization of New Zealand white rabbits. Rabbits were split into two cohorts. Cohort 1 received a prime immunization with 1×10⁷ Claudin18.2-transfected 293 Human Embryonic Kidney (HEK) cells in Complete Freund's adjuvant. 293 HEK cells were transfected with a eukaryotic expression vector encoding human full-length CLDN18.2 (FIG. 1, SEQ ID NO. 87). Following the Immunization Protocol in Table 1, rabbits then received 4 boosts at 7-day intervals containing 2-fold decreasing cell numbers in alternating Incomplete Freund's adjuvant and Alum CpG2007. After the 4^{th} immunization, rabbits received 7 boosts at 21-day intervals. All boosts contained 2-fold decreasing cell numbers in alternating Incomplete Freund's adjuvant and Alum CpG2007, except for days 49 and 70, where rabbits received 25 μg of recombinant human Claudin18.2 assembled into virus-like particles (VLPs) (Kactus Biosystems Cat. No. CLD-HE1822). The same immunization strategy was used for Cohort 2, except rabbits received 10-fold higher cell numbers and 50 μg recombinant human Claudin18.2 VLP. For both cohorts, whole peripheral blood was collected three weeks after each boost, and PBMC were isolated and stored in liquid nitrogen for sorting.

### PBMC Isolation:

Diluted blood was layered onto Lympholyte Cell Separation Media (Cedarlane, Cat. No. CL5050) and centrifuged at 2500 rpm for 30 minutes. Following centrifugation, the PBMC layer was carefully aspirated and washed. RBC remaining within the pellet were lysed using RBC Lysis Buffer (Qiagen, Cat. No. 158904). After lysis, cells were washed and resuspended in 10% DMSO in FBS at a cell concentration of ≤5×10⁷ cells per ml.

### Isolation of Rabbit B-Cells by FACS:

PBMC were thawed in immune cell media (ICM), then incubated with an antibody staining mix containing recombinant human CLDN18.2 VLP and reagents specific to rabbit B-cell and T-cell markers. Following incubation, PBMC were washed and incubated with fluorophore-specific microbeads. PBMC were enriched for B-cells by magnetic-activated cell sorting (MACS) using a QuadroMACS Separator magnet (Miltenyi Biotec, Cat. No. 130-090-976). Cells were then stained with efluor780 viability dye (ebioscience, Cat. No. 65-0865-14), washed, resuspended in buffer, and stored at 4 C until sorting by Fluorescence Activated Cell Sorting (FACS).

To sort B-cells, the appropriate compensation was performed using both sorter software and manual adjustments. Gates were adjusted to obtain live, IgG-positive rabbit B-cells. B-cells were sorted from the final gate as single cells into 96-well plates containing previously prepared B-cell culture media containing ICM, rabbit splenocyte conditioned media, and feeder cells. For each sort, up to 30 plates were sorted. Plates were incubated at 37° C. with 5% CO₂ for 12 days prior to screening.

### Example 2. Identification of Anti-Claudin18.2 Antibodies

### Antibody Screen by ELISA:

B-cell cultures were screened for IgG secretion and Claudin18.2 binding by direct sandwich ELISA. Plates were coated with 1 μg/ml anti-rabbit IgG Fc fragment (Jackson ImmunoResearch, Cat. No. 111-005-046) or Claudin18.2 VLP (Kactus Biosystems Cat. No. CLD-HE1822). Plates were washed three times with 1×PBST, then blocked with 2% BSA in DPBS. B-cell supernatant was harvested from culture plates and used for screening. B-cells remaining in the culture flask were stored in RNA Later (Invitrogen, Cat. No. AM7021) at −80° C. for RT-PCR amplification of antibody variable regions. After blocking, plates were washed as before, and B-cell supernatant was added to each plate at a 1:10 dilution. Following 1 hour incubation, plates were washed as before, then incubated with anti-Rb IgG secondary antibody conjugated to HRP (Jackson ImmunoResearch, Cat. No. 111-035-046) for 30 minutes. Plates were washed, then incubated with TMB substrate (ThermoFisher Scientific, Cat. No. 34029). Reactions were stopped by adding commercial stop solution (ThermoFisher Scientific, Cat. No. SS04) and plates were read using a spectrophotometer at 450 nm. Detection of rabbit IgG antibodies specific to Claudin18.2 were confirmed based off OD values exceeding the secondary only negative control.

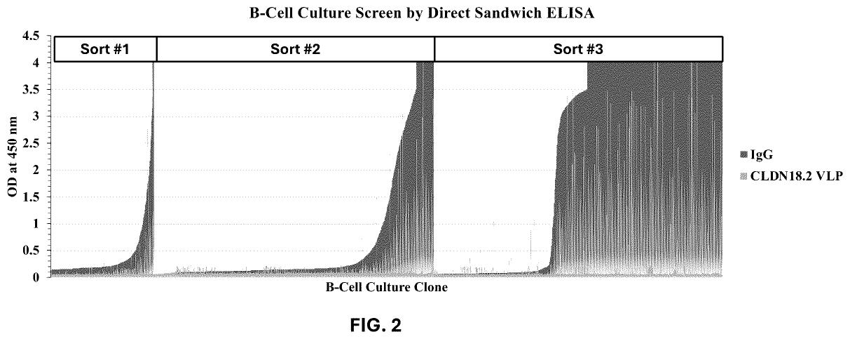

The rabbit IgG antibodies were grouped and plotted in FIG. 2. For Sort #1, 17.4% of B-cells sorted were IgG-positive. A total of 11 clones bound specifically to Claudin18.2 VLP. For Sort #2, 28.5% of B-cells sorted were IgG-positive. A total of 24 clones bound specifically to Claudin18.2 VLP. For Sort #3, 60% of B-cells sorted were IgG-positive. A total of 54 clones bound specifically to Claudin18.2 VLP.

### Antibody Screen by Flow Cytometry:

To confirm specificity, clones were tested for binding to Claudin18.2 and Claudin18.1 expressed on the surface of transfected 293 BEK. Detection of antibody binding to surface expressed Claudin18.2 was achieved by flow cytometry. 293 BEK cells were transiently transfected with eukaryotic expression vectors encoding human full-length Claudin18.2 and Claudin18.1 (FIG. 1, SEQ ID NO. 87 and 88, respectively). 293 BEK transfected with irrelevant DNA were used as a negative control. Two days following transfection, cells were collected, stained with cell tracker dye (ThermoFisher Scientific, Cat. No. C34565) and plated at ~1.5×10⁵ cells per well. B-cell supernatant was added to the cells at a 1:5 dilution and incubated for 1 hour at 4° C. Cells were washed, then incubated with Alexa Fluor 647 conjugated anti-rabbit IgG Fc secondary antibody (Jackson ImmunoResearch, Cat. No. 111-605-008) for 30 minutes at 4° C. After one wash, cells were resuspended in FACS Buffer. Binding was determined by flow cytometry using a BD Fortessa flow cytometer. Clones expressing antibodies that bound Claudin18.2, but not Claudin18.1 or negative control, were selected for molecular “rescue” of the antibody variable regions.

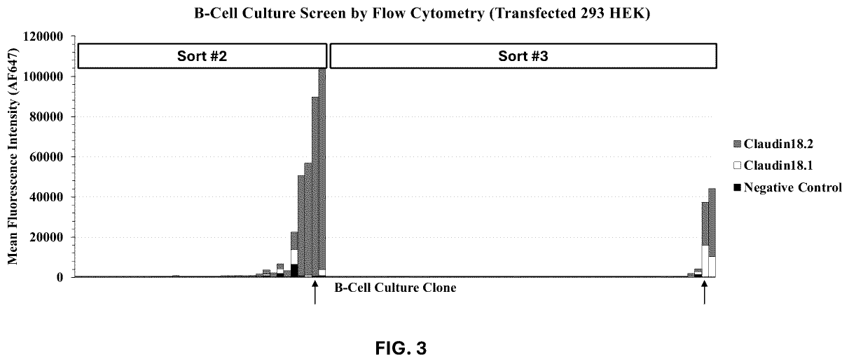

To identify anti-Claudin 18.2 antibodies, B-cell clones that bound surface expressed Claudin18.1 and Claudin18.2, but not the negative control, were selected for further screening. Representative clones bound Claudin18.2, but not Claudin18.1. The binding activity of B-cell supernatants obtained from Sorts #2 and #3 are shown (FIG. 3). One clone, which bound both Claudin18.1 and Claudin18.2, was expressed specifically for use as a screening reagent (5208H10). The disclosed antibodies selected are identified with black arrows (Sort #2: 5103F3, Sort #3: 5208H10).

### Example 3. Production of Rabbit/Human Chimeric Monoclonal Antibodies

### Amplification of Antibody Variable Regions:

B-cell clones identified by ELISA and flow cytometry were thawed from storage at −80° C. Heavy and light chain variable sequences were amplified by multiplex RT-PCR using primers specific to the leader sequence and constant region of rabbit IgG and rabbit kappa sequences. Amplicons were further amplified in a secondary PCR using nested primers that contained restriction sites. Heavy chain and light chain amplicons were cloned into an expression vector containing human IgG1 or human IgK, respectively. After sequence verification, expression plasmids were transiently co-transfected into 293 HEK cells to produce rabbit/human chimeric antibodies.

### Characterization of Chimeric Anti-Claudin18.2 Antibodies—Flow Cytometry:

Recombinant antibody supernatants were screened for binding to surface expressed Claudin18.2 by flow cytometry. ExpiCHO cells were transiently transfected with eukaryotic expression vectors encoding human full-length Claudin18.2 and Claudin18.1 (SEQ ID NO. 87 and 88, respectively). ExpiCHO cells transfected with irrelevant DNA were used as a negative control. Two days following transfection, cells were stained for flow cytometry as described for B-cell culture, however cells were incubated with chimeric antibodies serially diluted from 10 μg/ml to 0.0625 μg/ml.

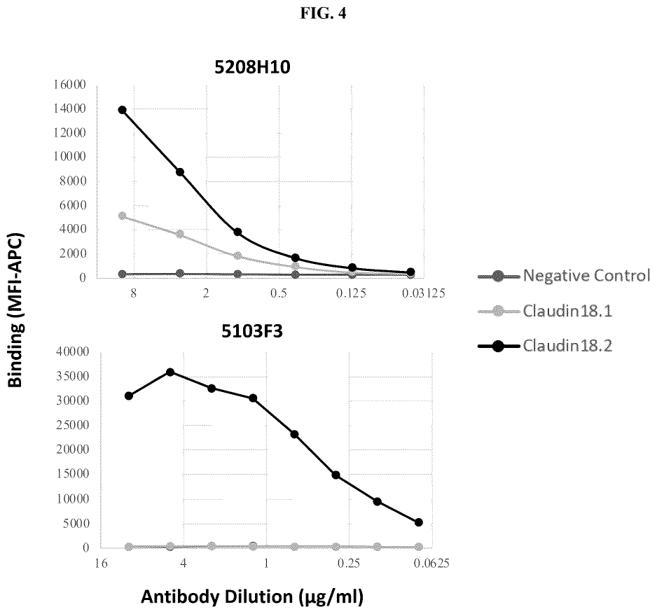

As shown in FIG. 4, representative antibodies will bind Claudin18.2 at high MFI but will not bind Claudin18.1 or the negative control. All chimeric antibodies bound to Claudin18.2. Claudin18.1 binding was not detected for all antibodies except 5208H10. 5208H10, a pan-Claudin18 binder, serves as a control for Claudin18.1 expression. Detection of both Claudin18.1 and Claudin18.2 on transiently transfected ExpiCHO supports the reported specificity of the anti-Claudin18.2 clones to Claudin18.2.

### Characterization of Chimeric Anti-Claudin18.2 Antibodies—OCTET Affinity:

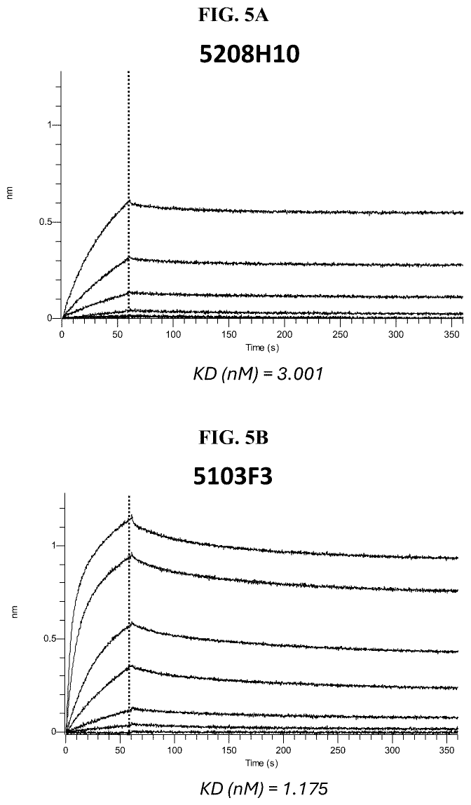

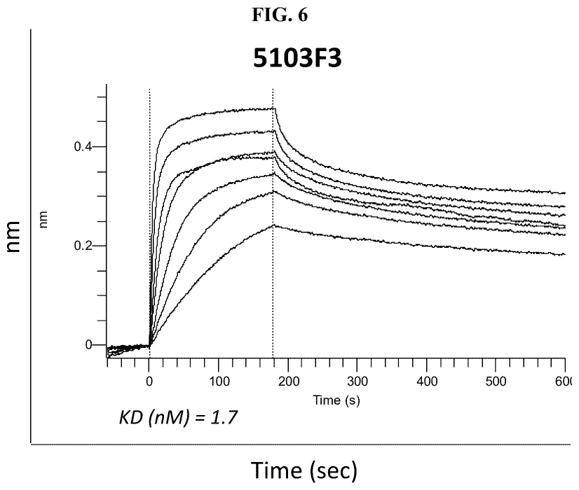

Recombinant antibody supernatants were validated for Claudin18.2 binding by bio-layer interferometry (also known as OCTET) using a ForteBio Octet Red 384 instrument. Antibodies (10 μg/ml) were captured onto anti-human Fc biosensors, then associated with recombinant human Claudin18.2-His full-length protein (Acro Biosystems, Cat. No. CL2-H82E3) diluted 1:3 from 300 nM. Sensors were moved to wells containing kinetics buffer, and the dissociation of the antibody from Claudin18.2 was measured. Curve fits and affinity calculations were analyzed using manufacturer-provided software. Chimeric antibodies bound recombinant human Claudin18.2 by bio-layer interferometry at high affinity with KD values in 1 to 3 nM range as shown in FIG. 5.

### Characterization of Chimeric Anti-Claudin18.2 Antibodies—OCTET Avidity:

Bio-layer interferometry (i.e., OCTET) was also used to evaluate chimeric antibody avidity to recombinant human Claudin18.2. Biotinylated recombinant human Claudin18.2 (10 μg/ml) was captured onto streptavidin biosensors, then associated with chimeric antibodies diluted 1:3 from 300 nM. Sensors were moved to wells containing kinetics buffer to analyze dissociation of the antibody/Claudin18.2 complex. Curve fits and avidity calculations were analyzed using manufacturer-provided software. The avidity for the chimeric antibody to recombinant human Claudin18.2 was within 1 to 2 nM range as shown in FIG. 6. Due to being only a control reagent, 5208H10 was not evaluated for avidity.

### Example 4. Production of Humanized Monoclonal Antibodies

### Humanization of Heavy and Light Chain Variable Regions:

The heavy and light chain variable regions for antibodies were humanized using the ‘Predict Humanizing Mutations’ algorithm in Discovery Studio. Methods chosen for humanization included best single mutation (BSM), frequent residue substitution (FRS), or germline graft (GS). DNA sequences encoding the humanized antibody VH and VL regions were cloned into mammalian expression vectors containing human IgG1/kappa constant regions using gene fragments (Genewiz). Due to its use as a research tool, the pan-Claudin18 binder (5208H10) was not humanized.

### Protein Stability by UPLC-SEC:

Immediately after first-step protein A purification, antibodies were analyzed by analytical SEC using Waters Acquity UPLC H-Class with ACQUITY UPLC® Protein BEH SEC 200 Å, 4.6 mm×150 mm, 1.7 μm column. PBS (125 mM sodium phosphate, 137 mM sodium chloride, pH 6.8) was used as mobile phase for 10-minute runs at 0.3 ml/min, injecting 10 μg protein. Proteins were buffer-exchanged into 25 mM sodium acetate, 125 mM NaCl, 10% sucrose, pH 5.5. Antibody samples were injected into the same column after approximately three weeks and six weeks after the initial purification to test stability over time. UPLC-SEC traces were integrated into three categories: % POI (protein of interest), % HMW (high molecular weight), and % LMW (low molecular weight). The change in each species (POI, HMW, LMW) was calculated based on the difference in the integrated area between the first and last stability time points. As summarized in Table 2, the data demonstrate that most humanized anti-Claudin 18.2 antibodies were stable for at least four to six weeks under these conditions, showing negligible change in the protein of interest species over time.

### Example 5. Characterization of Humanized Antibodies

### Flow Cytometry and OCTET:

Humanized antibodies were screened by Flow Cytometry and OCTET as described earlier. For comparison, anti-CLDN18.2 antibodies included: the humanized antibody, 5103F3 BSM; the pan-Claudin18 binder antibody, 5208H10 (SI-64CH5); Zolbetuximab (SI-64C8); and other positive control antibodies, SI-64C9, SI-64C10, and SI-64C11. Specifically, the anti-CLDN18.2 antibodies were expressed by transiently transfected ExpiCHO and the levels of expression were scored (Table 3). To evaluate the binding affinity and avidity of anti-CLDN antibodies, the Bio-Layer Interferometry was used to measure KD values as shown in Table 4. The KD values of anti-CLDN18.2 antibodies were clustered between 0.7 nM and 1.45 nM or within a range of 2-fold for affinity, whereas the KD values for avidity were between 0.22 nM and 5.31 nM or a more than 20× improvement in functional affinity of 5103F3 BSM over Zolbetuximab.

### Validation of Claudin18.2 Specificity by Flow Cytometry:

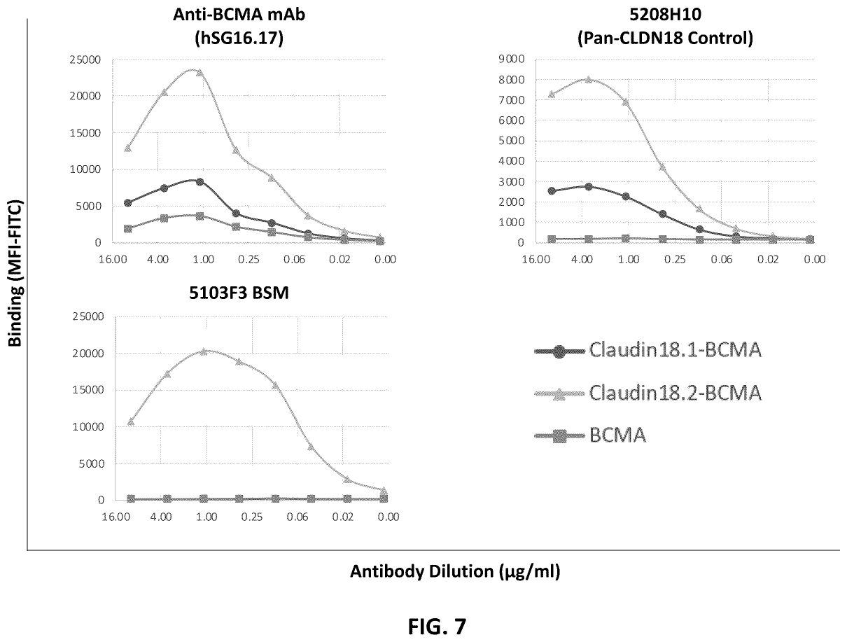

To further confirm the specificity of representative antibodies to Claudin18.2 and not Claudin18.1, BCMA-Claudin18 fusion proteins were expressed in transiently transfected ExpiCHO. Flow cytometry methods were followed as described previously. BCMA expression was detected using an anti-BCMA antibody. Detection of BCMA confirmed expression of BCMA-Claudin18.1 and BCMA-Claudin18.2 as shown in FIG. 7. Expression was also confirmed using the pan-Claudin18 binder, 5208H10. Both controls confirmed that lack of binding by humanized antibodies was not due to lack of expression. Clone 5103F3 bound BCMA-Claudin18.2, but not BCMA-Claudin18.1, further supporting specificity to Claudin18.2.

### Validation of Claudin18.2 Specificity by ELISA:

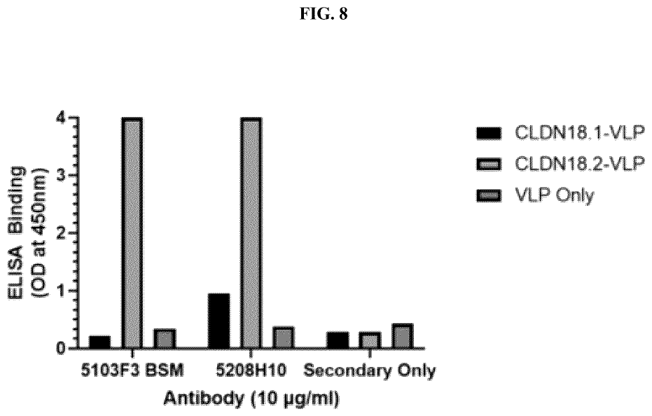

Specificity of representative antibody binding to Claudin18.2 was further validated by direct sandwich ELISA as previously described, with the following changes. Plates were coated with 1 μg/ml recombinant protein VLPs: Claudin18.2 VLP (Acro Biosystems, Cat. No. CL2-H52P7), Claudin18.1 VLP (Acro Biosystems, Cat. No. CL1-H52P3), or empty VLP isotype control (Acro Biosystems, Cat. No. VLP-N5213). Antibodies were tested at 10 μg/ml, and binding was detected using an anti-Hu IgG secondary antibody conjugated to HRP (Jackson ImmunoResearch, Cat. No. 309-035-003). The result shows that 5103F3 BSM bound CLDN18.2-VLP, but not CLDN18.1-VLP or VLP-only control (FIG. 8). The pan-Claudin18 binder, 5208H8, bound both CLDN18.1 and CLDN18.2, but not VLP-only control. Taken together, the flow cytometry and ELISA data demonstrate the specificity of 5103F3 BSM to Claudin18.2.

### Antibody Binning by Bio-Layer Interferometry (OCTET):

The epitopes of antibodies were binned by Bio-Layer Interferometry (i.e., OCTET) In-Tandem Assay using a ForteBio OCTET Red 384 instrument. Biotinylated recombinant human Claudin18.2-His full-length protein (Acro Biosystems, Cat. No. CL2-H82E3, 10 μg/ml) was captured onto streptavidin-conjugated biosensors, then associated with 10 μg/ml of antibody (i.e., 1^{st} association). Sensors were then moved to wells containing a different antibody (i.e., 2^{nd} association). Using the instrument software, antibodies were binned based off hierarchical clustering of the change in response from the 1^{st} association to the 2^{nd} association. To determine blocking or nonblocking antibody pairs, a threshold was set at the appropriate self-binding signal. Antibodies that blocked binding were clustered into the same bin and considered to have similar epitopes. Antibodies that did not block binding were clustered into separate bins and considered to have different epitopes.

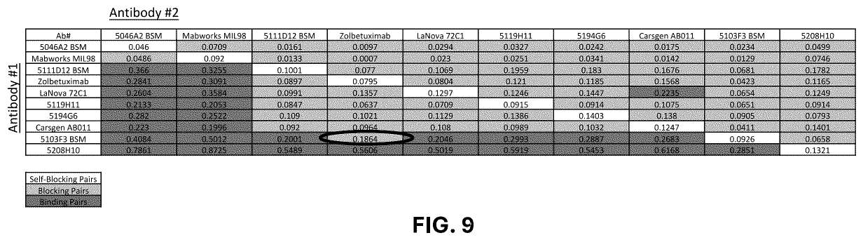

The hierarchical clustering of disclosed antibodies, candidate antibodies, and positive control comparator antibodies (SI-64C8 to SI-64C11) are shown in the binning matrix table (FIG. 9). Multiple candidate antibodies blocked binding of the comparator antibodies, indicating that these clones have similar epitopes to those that are already in development. However, the disclosed clone, 5103F3 BSM, showed unidirectional binding activity to all antibodies, including most comparator antibodies. When bound to Claudin18.2 as Antibody #1, 5103F3 BSM did not block binding of the candidate or comparator antibodies, except SI-64C8. When bound as Antibody #2, 5103F3 BSM was blocked by all antibodies. After clustering, 5103F3 BSM was organized into a bin that was distinguishable from all candidate and comparator antibodies (circle). This suggests that 5103F3 BSM has a unique epitope-blocking profile when compared to other blocking antibodies.

### Example 6. Anti-Claudin18.2 Antibody-Dependent Cytotoxicity

### Complement-Dependent Cytotoxicity (CDC):

The Fc-mediated CDC activity of antibody-based therapeutics can be compared in vitro to determine if there are features of an antibody that enable a greater potential therapeutics mode of action. To compare the CDC function of 5103F3 BSM and Zolbetuximab, Chinese hamster ovarian cells (CHO) were transduced to express Claudin 18.2 tagged to a fluorescent reporter. Target cells CHO-Claudin18.2 were treated with 5103F3 BSM and Zolbetuximab in the presence and absence of Normal Human Serum (NHS) to evaluate CDC in 2 hours. Cells were treated with antibody in the assay ranging from 6.4 pM to 500 nM across a 5-fold dilution curve. CDC was evaluated by uptake of membrane impermeant fluorescent dye measured using FACS.

The CDC activity of Zolbetuximab was detectable in this assay system. 5103F3 BSM showed equivalent CDC activity. This is consistent with equivalent binding activity over the duration of the assay and access to assemble the CDC multimeric compliment cascade.

### Antibody-Dependent Cell-Mediated Cytotoxicity (ADCC:

The Fc-mediated ADCC activity of antibody-based therapeutics can be compared in vitro to determine if there are features of an antibody that enable a greater potential therapeutics mode of action. In this testing system, a low ADCC responses may be due to the available antigen expression level over the duration of the assay. As a result, different binding properties, or antibody internalization may have an impact on the ADCC activity¹⁰.

To test this, human primary NK (Natural Killer) cells were enriched from whole blood by negative selection, followed by density gradient centrifugation to become “effector” cells. The CHO cells that were transduced to express Claudin18.2 tagged to a fluorescent reporter were target cells. The CHO-Claudin18.2 target cells were treated with 5103F3 BSM, Zolbetuximab or rituximab, ranging from 100 nM to 0.00001 nM in a 10-fold serial dilution. NK cells were added at a 5:1 E:T (effector:target) ratio to evaluate ADCC within the 24-hour assay timeframe. ADCC was evaluated by loss of CHO-Claudin 18.2 cell reporter signal using time-series fluorescent microscopy.

The ADCC activity of Zolbetuximab was detectable in this assay system and the CD20 specific Rituximab did not demonstrate ADCC activity. 5103F3 BSM showed significantly different pharmacokinetics characterized by extending the ADCC activity. The enhanced ADCC activity of 5103F3 BSM compared to Zolbetuximab is consistent with greater antigen availability over the course of the testing allowing for greater NK cell engagement and cytolytic function.

### Example 7. Internalization of Anti-CLDN18.2 Antibodies and Lysosomal Trafficking

For many antibody-drug conjugate (ADC) drugs, a key step of the mechanism of action involves internalization¹¹. Following antibody binding, internalization can facilitate the uptake of cytotoxic payload and trafficking to cancer cell lysosomes where lysosomes act as recycling centers within cells. Depending on the types of cytotoxic payload, cell lysis follows different mechanisms of action. Claudin 18.2 is a tight junction protein normally expressed on gastric epithelial cells and has additionally been detected in gastric, pancreatic, biliary tract and colorectal cancers¹². Thus, the mechanism of internalization and trafficking to lysosomes by anti-Claudin18.2 may be the same, but the efficacy may depend on the antibody binding.

The anti-Claudin18.2 antibodies, 5103F3 BSM and Zolbetuximab, were labeled with FabFluor-pH red, an acid-sensitive fluorescent reagent that allows quantification of the trafficking of internalized proteins to acidic lysosomes. The fluorescent signal of FabFluor-pH red is dependent on the low pH environment of lysosomes. In this way, the accumulation of internalized antibodies into lysosomal compartments may be quantified over time using time-series fluorescent microscopy. NUGC-4 cell line was thawed and cultured in RPMI media supplemented with Pen/Strep and 10% FBS. Cells were seeded at 10,000 cells/well in 50 μL of culture in 96-well round bottom ULA plates (Corning). Vehicle buffer, 5103F3 BSM, and Zolbetuximab were labeled with FabFluor-pH according to the manufacturer's instructions. Fifty microliters of FabFluor-pH labeled antibodies or control treatments were added to plated cells at 2× (10 nM or 20 nM) concentrations for 100 μl final assay volume/well. Cells were cultured in an Incucyte S3 live cell imager (Sartorius) in a 37° C., 5% CO2 humidified incubator for 16 hours.

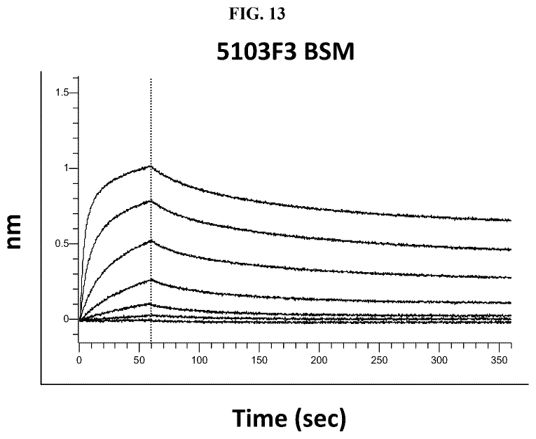

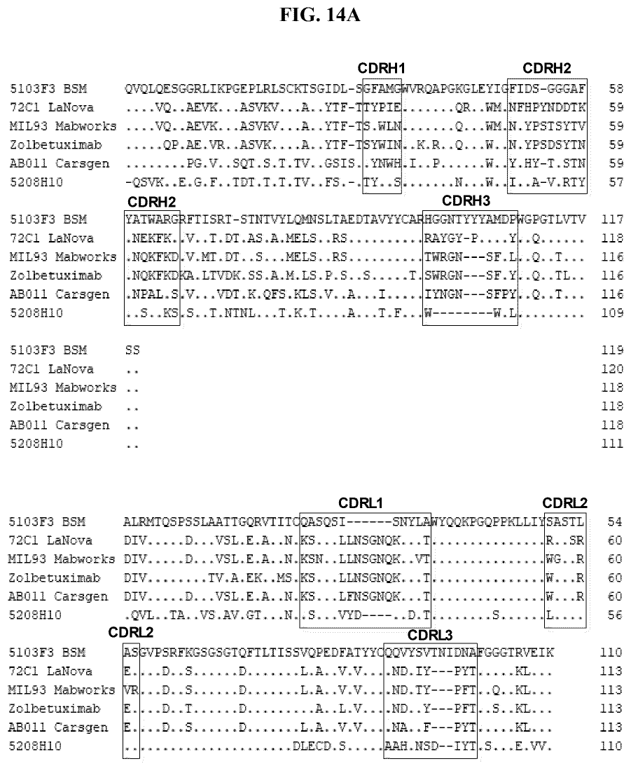

Using time-series live-cell fluorescence microscopy, the internalization and trafficking signals were readily detectable for both 5103F3 BSM and Zolbetuximab at either 5 nM or 10 nM concentrations. The signals were automatically counted and plotted over time. The result shows that 5103F3 BSM was internalized more efficiently and trafficked more signals (indicative of cytotoxic payload in case of ADC) to lysosomes than Zolbetuximab (FIG. 13).

### Example 8. Characterization of 5103F3 BSM

### Cross-Species Binding to Mouse and Rat Claudin18.2:

Humanized anti-Claudin18.2 antibodies were analyzed for cross-species binding to mouse and rat Claudin18 by flow cytometry. ExpiCHO were transiently transfected with plasmids expressing mouse or rat Claudin18.1 and Claudin18.2. Binding of surface-expressed Claudin18 was determined using previously described methods. Table 5 shows that the antibody 5103F3 bound mouse and rat Claudin18.2, but not Claudin18.1. Due to high sequence similarity to human (98.9% for both extracellular domains), cynomolgus monkey Claudin18 was not included in the assay. The binding of 5103F3 BSM to mouse Claudin18.2 was confirmed by OCTET, with a binding affinity of 1.73 nM (FIG. 13).

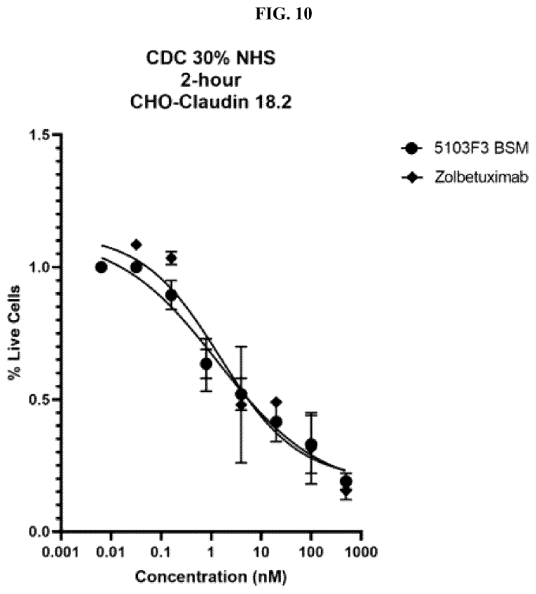

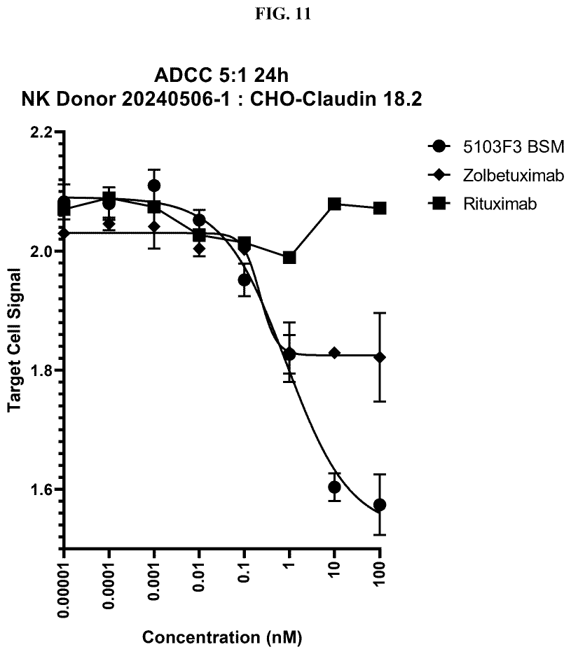

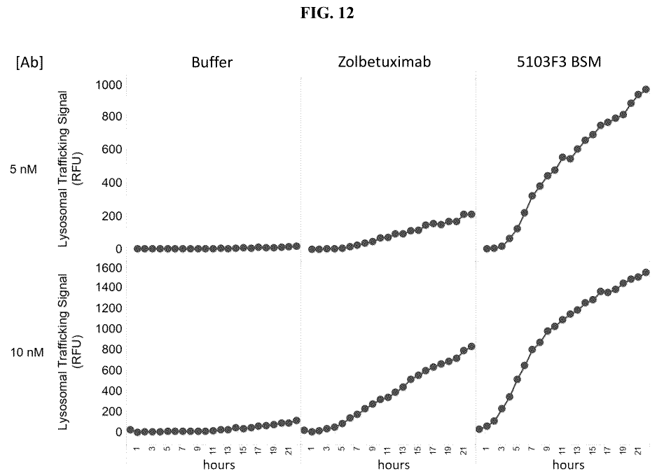

Zolbetuximab is a human/mouse chimeric antibody therapeutics for treating patients with gastric cancer in combination with chemotherapy. In contrast, 5103F3 BSM is a humanized antibody. With a higher degree of humanness, 5103F3 BSM may exert improved therapeutic effect by reducing immunogenicity or other unwanted side effects after administration. In addition, 5103F3 BSM recognized a distinct epitope bin from isoform 2 of Claudin-18 when compared to Zolbetuximab (SI-64C8) and other anti-CLDN18.2 antibodies, including AB011 (SI-64C10), LM-102 (SI-64C9), and MIL93 (SI-64C11) (FIG. 9). This binding character of 5103F3 BSM may lead to unique binding specificity, clustering, internalization, and other biological activities. Indeed, 5103F3 BSM and Zolbetuximab have exhibited similar CDC but different ADCC activities (FIGS. 10 and 11), as well as different pharmacokinetics in internalization and lysosomal trafficking (FIG. 12).

### Sequence Alignment of Lead and Comparator Antibodies:

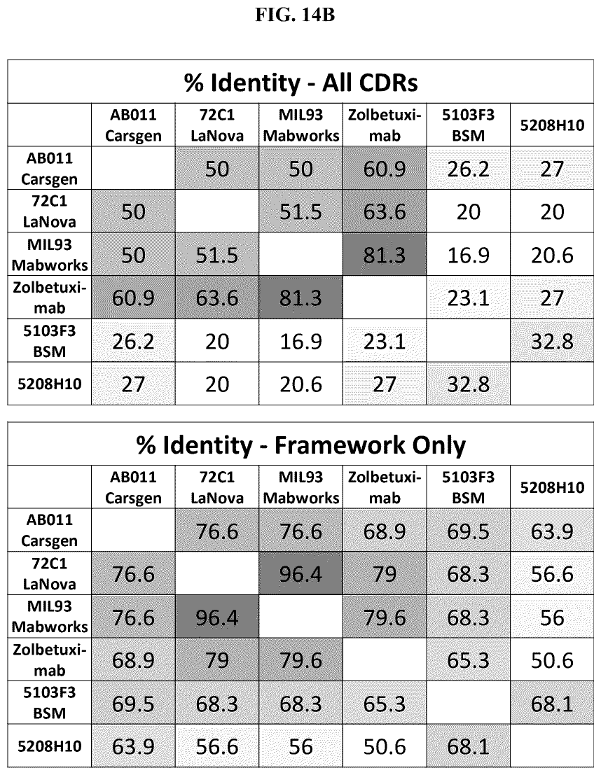

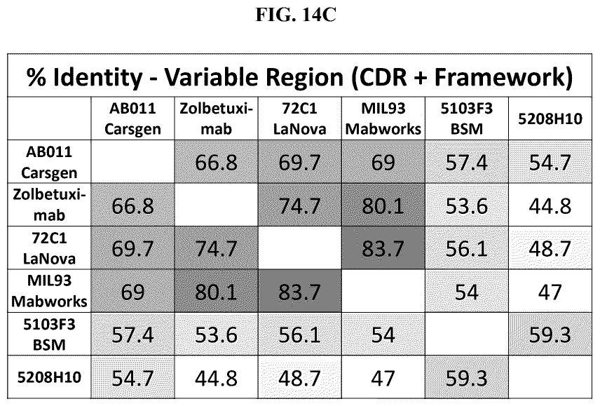

To investigate the structural basis of 5103F3 BSM's unique characters, the coding sequences 5103F3 BSM, 5208H10, and comparator antibodies were aligned in the Kabat numbering scheme using Geneious Prime bioinformatics software and shown in FIG. 14. The complementarity-determining regions (CDRs) of heavy and light chains were aligned across all antibodies (boxed in upper and lower panels of FIG. 14A). The percentage identity for all CDRs only, the framework regions only, and the entire variable region were calculated for comparison in FIGS. 14B and 14C. Of aligned CDR sequences, 5103F3 BSM shared 17-28% identity with each comparator whereas each comparator shared 50-82% identify with another comparator. Of all framework regions, 5103F3 BSM shared up to 70% identity with each comparator. Using a two-tailed, one-sample T-test, the percentage identity of 5103F3 BSM to a given comparator was then compared to the average of percentage identities among the remaining comparators. The differences were significant (P<0.05) in both CDRs and the full variable regions but not in all framework regions (P>0.05) (Table 6). The significant differences in the amino acid sequences encoding the primary structure of CDRs underlie the characteristic features of 5103F3 BSM as compared to comparator antibodies in epitope binding (FIG. 9) and consequently, the biological activity.

In summary, 5103F3 BSM is a humanized anti-CLDN18.2 antibody with high affinity, avidity, and specificity to Claudin18.2, whereas 5208H10 is a pan-Claudin18 binder capable of binding both Claudin18.1 and Claudin 18.2. The pair of antibodies are useful for developing antibody therapy as well as diagnosing Claudin expression in different forms of cancer. Additionally, due to the significant technical advantages of 5103F3 BSM over other antibodies in the similar category including its high selectivity of Claudin18.2, 5103F3 BSM provides significant therapeutical advantage for treating cancers with overexpression of Claudin18.2.

### REFERENCE

1. Stomach cancer statistics|WCRF International (https://www.wcrf.org/cancer-trends/stomach-cancer-statistics/)
2. American Cancer Society: Cancer Facts and Figures 2022. American Cancer Society, 2022. (Cancer Facts & Figures 2022)
3. Pancreatic cancer statistics—WCRF International (https://www.wcrf.org/cancer-trends/pancreatic-cancer-statistics/)
4. Key Statistics for Pancreatic Cancer (https://www.cancer.org/cancer/pancreatic-cancer/about/key-statistics.html)
5. Cao W, Xing H, Li Y, Tian W, Song Y, Jiang Z, Yu J. Claudin18.2 is a novel molecular biomarker for tumor-targeted immunotherapy. Biomark Res. 2022 May 31; 10(1):38. doi: 10.1186/s40364-022-00385-1. PMID: 35642043; PMCID: PMC9153115.
6. Sahin U, Koslowski M, Dhaene K, Usener D, Brandenburg G, Seitz G, Huber C, Türeci &. Claudin-18 splice variant 2 is a pan-cancer target suitable for therapeutic antibody development. Clin Cancer Res. 2008 Dec. 1; 14(23):7624-34. doi: 10.1158/1078-0432.CCR-08-1547. PMID: 19047087.
7. Niimi T, Nagashima K, Ward J M, Minoo P, Zimonjic D B, Popescu N C, Kimura S. claudin-18, a novel downstream target gene for the T/EBP/NKX2.1 homeodomain transcription factor, encodes lung- and stomach-specific isoforms through alternative splicing. Mol Cell Biol. 2001 November; 21(21):7380-90. doi: 10.1128/MCB.21.21.7380-7390.2001. PMID: 11585919; PMCID: PMC99911.
8. Li J. Targeting claudins in cancer: diagnosis, prognosis and therapy. Am J Cancer Res. 2021 Jul. 15; 11(7):3406-3424. PMID: 34354852; PMCID: PMC8332862.
9. Türeci O, Koslowski M, Helftenbein G, Castle J, Rohde C, Dhaene K, Seitz G, Sahin U. Claudin-18 gene structure, regulation, and expression is evolutionary conserved in mammals. Gene. 2011 Aug. 1; 481(2):83-92. doi: 10.1016/j.gene.2011.04.007. Epub 2011 May 4. PMID: 21571049.
10. Garvin D, Stecha P, Gilden J, Wang J, Grailer J, Hartnett J, Fan F, Cong M, Cheng Z J. Determining ADCC Activity of Antibody-Based Therapeutic Molecules using Two Bioluminescent Reporter-Based Bioassays. Curr Protoc. 2021 November; 1(11):e296.
11. Yu et al. Frontiers Molecular Biosciences 2022.
12. Yong Hong et al. Trans Cancer Res 2020.

### TABLES

TABLE 1
Immunization protocol for generating anti-CLDN18.2 antibody - Cohort 1 and 2

| | | Dose for | Dose for | | |
|---|---|---|---|---|---|
| Day Post- | | Cohort 1 | Cohort 2 | | Whole |
| Immunization | Immunogen | (μg or cell no.) | (μg or cell no.) | Adjuvant | Bleed |
| 0 | 293HEK- | 1 × 10⁷ cells | 1 × 10⁸ cells | Complete | No |
| | CLDN18.2 | | | Freund's | |
| 7 | 293HEK- | 1 × 10⁷ cells | 1 × 10⁸ cells | Incomplete | No |
| | CLDN18.2 | | | Freund's | |
| 14 | 293HEK- | 5 × 10⁶ cells | 5 × 10⁷ cells | Alum | No |
| | CLDN18.2 | | | CpG2007 | |
| 21 | 293HEK- | 5 × 10⁶ cells | 5 × 10⁷ cells | Incomplete | No |
| | CLDN18.2 | | | Freund's | |
| 28 | 293HEK- | 2.5 × 10⁶ cells | 2.5 × 10⁷ cells | Alum | Yes |
| | CLDN18.2 | | | CpG2007 | |
| 49 | Rec. Hu | 25 μg | 50 μg | Incomplete | Yes |
| | CLDN18.2 | | | Freund's | |
| 70 | Rec. Hu | 25 μg | 50 μg | Alum | Yes |
| | CLDN18.2 | | | CpG2007 | |
| 91 | 293HEK- | 2.5 × 10⁶ cells | 2.5 × 10⁷ cells | Incomplete | Yes |
| | CLDN18.2 | | | Freund's | |
| 112 | 293HEK- | 2.5 × 10⁶ cells | 2.5 × 10⁷ cells | Alum | Yes |
| | CLDN18.2 | | | CpG2007 | |
| 133 | 293HEK- | 2.5 × 10⁶ cells | 2.5 × 10⁷ cells | Incomplete | Yes |
| | CLDN18.2 | | | Freund's | |
| 154 | 293HEK- | 1.25 × 10⁶ cells | 1.25 × 10⁷ cells | Alum | No |
| | CLDN18.2 | | | CpG2007 | |
| 175 | 293HEK- | 1.25 × 10⁶ cells | 1.25 × 10⁷ cells | Incomplete | Yes |
| | CLDN18.2 | | | Freund's | |

TABLE 2
Stability of humanized anti-Claudin18.2 antibodies at Time 0 and Time 1 (upper
panel), and Time 2 and Change (lower panel).

| | Time 0 | | | | Time 1 | | | |
|---|---|---|---|---|---|---|---|---|
| Antibody | Date | % POI | % HMW | % LMW | Date | % POI | % HMW | % LMW |
| 5046A2 | Sep. 10, 2021 | 93.98 | 0.86 | 5.16 | Sep. 30, 2021 | 94.29 | 0.5 | 5.21 |
| BSM | | | | | | | | |
| 5046A2 | Sep. 10, 2021 | 100 | 0 | 0 | Sep. 30, 2021 | 100 | 0 | 0 |
| FRS | | | | | | | | |
| 5103F3 | Sep. 10, 2021 | 100 | 0 | 0 | Sep. 30, 2021 | 100 | 0 | 0 |
| BSM | | | | | | | | |
| 5103F3 | Oct. 22, 2021 | 92.48 | 7.52 | 0 | Nov. 11, 2021 | 93.74 | 6.26 | 0 |
| FRS | | | | | | | | |
| 5111D12 | Sep. 10, 2021 | 100 | 0 | 0 | Sep. 30, 2021 | 100 | 0 | 0 |
| BSM | | | | | | | | |
| 5194G6 | Dec. 1, 2021 | 99.54 | 0.46 | 0 | Dec. 20, 2021 | 99.44 | 0.56 | 0 |
| BSM | | | | | | | | |
| 5194G6 | Oct. 28, 2021 | 99.13 | 0 | 0 | Nov. 11, 2021 | 98.69 | 1.31 | 0 |
| FRS | | | | | | | | |
| 5194G6 | Oct. 28, 2021 | 97.2 | 3.18 | 0 | Nov. 11, 2021 | 99.86 | 0.14 | 0 |
| GS | | | | | | | | |
| | | | | | Change | | | |
| | Time 2 | | | | | Δ | Δ | Δ |
| Antibody | Date | % POI | % HMW | % LMW | Weeks | % POI | % HMW | % LMW |
| 5046A2 | Oct. 21, 2021 | 99.08 | 0.92 | 0 | 5.9 | 5.1 | 0.06 | -5.16 |
| BSM | | | | | | | | |
| 5046A2 | Oct. 21, 2021 | 100 | 0 | 0 | 5.9 | 0 | 0 | 0 |
| FRS | | | | | | | | |
| 5103F3 | Oct. 21, 2021 | 100 | 0 | 0 | 5.9 | 0 | 0 | 0 |
| BSM | | | | | | | | |
| 5103F3 | Nov 30, 2021 | 93.32 | 6.68 | 0 | 5.6 | 0.84 | −0.84 | 0 |
| FRS | | | | | | | | |
| 5111D12 | Oct. 21, 2021 | 100 | 0 | 0 | 5.9 | 0 | 0 | 0 |
| BSM | | | | | | | | |
| 5194G6 | Jan 6, 2022 | 99.26 | 0.74 | 0 | 5.1 | −0.28 | 0.28 | 0 |
| BSM | | | | | | | | |
| 5194G6 | Nov 30, 2021 | 97.82 | 2.18 | 0 | 4.7 | −1.31 | 2.18 | 0 |
| FRS | | | | | | | | |
| 5194G6 | Nov 30, 2021 | 99.81 | 0.19 | 0 | 4.7 | 2.61 | −2.99 | 0 |
| GS | | | | | | | | |

TABLE 3
Anti-CLDN18.2 antibodies and the levels
of expression in CHO cells.

| | | | ExpiCHO |
|---|---|---|---|
| Antibody | Antibody | Antibody | MFI |
| Code | Name | Background | (3 ug/ml) |
| SI-64M1 | 5103F3 BSM | Humanized Rabbit | 21323 |
| SI-64CH5 | 5208H10 | Human/Rabbit Chimeric | 8767 |
| SI-64C8 | Zolbetuximab | Human/Mouse Chimeric | 10761 |
| SI-64C9 | 72C1 - LaNova | Unknown | 25743 |
| SI-64C10 | AB011 - Carsgen | Humanized | Not available |
| SI-64C11 | MIL93 - Mabworks | Humanized | Not available |

TABLE 4
OCTET affinity and avidity of anti-CLDN18.2 antibodies.

| | OCTET Affinity | | | OCTET Avidity | | |
|---|---|---|---|---|---|---|
| | KD | kon | kdiss | KD | kon | kdiss |
| | (M) | (1/Ms) | (1/s) | (M) | (1/Ms) | (1/s) |
| SI- | 1.45E− | 5.23E+ | 7.61E− | 2.16E− | 3.79E+ | 8.17E− |
| 64M1 | 09 | 05 | 04 | 10 | 06 | 04 |
| SI- | 3.00E− | 9.50E+ | 2.85E− | Not Tested | | |
| 64CH5 | 09 | 04 | 04 | | | |
| SI- | 1.59E− | 2.18E+ | 3.46E− | 5.31E− | 5.81E+ | 3.08E− |
| 64C8 | 09 | 05 | 04 | 09 | 05 | 03 |
| SI- | 1.87E− | 3.49E+ | 6.53E− | 1.73E− | 6.54E+ | 1.13E− |
| 64C9 | 09 | 05 | 04 | 09 | 05 | 03 |
| SI- | 4.35E− | 4.74E+ | 2.06E− | 4.56E− | 5.38E+ | 2.45E− |
| 64C10 | 10 | 05 | 04 | 10 | 05 | 04 |
| SI- | 7.14E− | 4.60E+ | 3.28E− | 1.76E− | 1.09E+ | 1.93E− |
| 64C11 | 10 | 05 | 04 | 09 | 06 | 03 |

TABLE 5
The binding specificity of humanized clones
and controls to mouse and rat CLDN18.2.

| Antibody | Mouse | | Rat | |
|---|---|---|---|---|
| Code | CLDN18.1 | CLDN18.2 | CLDN18.1 | CLDN18.2 |
| SI-64M1 | 118 | 11759 | 119 | 8743 |
| SI-64CH5 | 89.1 | 210 | 126 | 2655 |
| SI-64C8 | 162 | 8024 | 144 | 5509 |
| SI-64C9 | 117 | 7729 | 262 | 5147 |

TABLE 6
Comparison of coding sequences for CDRs and
framework of the variable region between 5103F3
BSM and other anti-CLDN18.2 antibodies.

| | | Variable | CDR | Framework |
|---|---|---|---|---|
| | | Region | Only | Only |
| Zolbetuximab (SI-64C8) | | | | |
| SI-64C9, SI-64C10, | Average % | 73.87 | 68.6 | 75.83 |
| SI-64C11 | Identity (SD) | (±6.7) | (±11.1) | (±6.0) |
| 5103F3 BSM | % Identity: | 53.6 | 23.1 | 65.3 |
| P-Value | Two-Tailed, | 0.0344 | 0.0192 | 0.0936 |
| | One-Sample | | | |
| | T-Test | | | |
| P < 0.05 | Significantly | Yes | Yes | No |
| | Different | | | |
| 72C1-LaNova (SI-64C9) | | | | |
| SI-64C8, SI-64C10, | Average % | 76.03 | 55.03 | 84 |
| SI-64C11 | Identity (SD) | (±7.1) | (±7.5) | (±10.8) |
| 5103F3 BSM | % Identity: | 56.1 | 20 | 68.3 |
| P-Value | Two-Tailed, | 0.0397 | 0.0148 | 0.1282 |
| | One-Sample | | | |
| | T-Test | | | |
| P < 0.05 | Significantly | Yes | Yes | No |
| | Different | | | |
| AB011 - Carsgen (SI-64C10) | | | | |
| SI-64C8, SI-64C9, | Average % | 68.5 | 53.63 | 74.03 |
| SI-64C11 | Identity (SD) | (±1.5) | (±6.3) | (±4.44) |
| 5103F3 BSM | % Identity: | 57.4 | 26.2 | 69.5 |
| P-Value | Two-Tailed, | 0.0061 | 0.0171 | 0.2194 |
| | One-Sample | | | |
| | T-Test | | | |
| P < 0.05 | Significantly | Yes | Yes | No |
| | Different | | | |
| MIL93 - Mabworks (SI-64C11) | | | | |
| SI-64C8, SI-64C9, | Average % | 77.6 | 60.93 | 84.2 |
| SI-64C10 | Identity (SD) | (±7.7) | (±17.7) | (±10.7) |
| 5103F3 BSM | % Identity: | 54 | 16.9 | 68.3 |
| P-Value | Two-Tailed, | 0.0334 | 0.0496 | 0.123 |
| | One-Sample | | | |
| | T-Test | | | |
| P < 0.05 | Significantly | Yes | Yes | No |
| | Different | | | |

| | | Variable | Nucleic Acid | Amino Acid |
|---|---|---|---|---|
| Antibody | | Region | SEQ ID NO. | SEQ ID NO. |
| 5103F3 | Rabbit | VH | 1 | 2 |
| | | VL | 3 | 4 |
| 5103F3 | Humanized | VH | 5 | 6 |
| BSM | | VL | 7 | 8 |
| 5103F3 | Chimeric | VH | 9 | 10 |
| FRS | | VL | 11 | 12 |
| 5208H10 | Rabbit | VH | 13 | 14 |
| | | VL | 15 | 16 |
| 5046A2 | Rabbit | VH | 17 | 18 |
| | | VL | 19 | 20 |
| 5046A2 | Humanized | VH | 21 | 22 |
| BSM | | VL | 23 | 24 |
| 5046A2 | Chimeric | VH | 25 | 26 |
| FRS | | VL | 27 | 28 |
| 5111D12 | Rabbit | VH | 29 | 30 |
| | | VL | 31 | 32 |
| 5111D12 | Humanized | VH | 33 | 34 |
| BSM | | VL | 35 | 36 |
| 5194G6 | Rabbit | VH | 37 | 38 |
| | | VL | 39 | 40 |
| 5194G6 | Humanized | VH | 41 | 42 |
| BSM | | VL | 43 | 44 |
| 5194G6 | Chimeric | VH | 45 | 46 |
| FRS | | VL | 47 | 48 |
| 5194G6 | GS | VH | 49 | 50 |
| GS | | VL | 51 | 52 |
| 5119H11 | Rabbit | VH | 53 | 54 |
| | | VL | 55 | 56 |

| | VH | | | VL | | |
|---|---|---|---|---|---|---|
| Antibody | CDRH1 | CDRH2 | CDRH3 | CDRH1 | CDRH2 | CDRH3 |
| 5103F3 | 57 | 58 | 59 | 60 | 61 | 62 |
| 5208H10 | 63 | 64 | 65 | 66 | 67 | 68 |
| 5046A2 | 69 | 70 | 71 | 72 | 73 | 74 |
| 5111D12 | 75 | 76 | 77 | 78 | 79 | 80 |
| 5119H11 | 81 | 82 | 83 | 84 | 85 | 86 |

### SEQUENCE LISTING

>Sequence ID 1: 5103F3 Rabbit VH Nucleic Acid Sequence
CAGTCGGTGGAGGAGTCCGGGGGTCGCCTGGTCACGCCTGGGACAGCCCTGACACTCACCTGCA
CAGTCTCTGGATTCTCCCTCAGTAGCTATTCAATGGGCTGGGTCCGCCAGGCTCCAGGGAAGGG
GCTGGAGTGGATCGGATATCTTAATACTGGTGGTAGCACATACTACGCGAGCTGGGTGAATGGT
CGATTCACCATCTCCAAAACCTCGTCGACCACGGTGGATCTGAAAATCACCAGTCCGACAACCG
AGGACACGGCCACCTATTTCTGTACCAGGGCCGGGGGTGTTAGTAGCAATATGGGCTTTAACAT
CTGGGGCCAAGGGACCCTGGTCACCGTCTCGAGC
>Sequence ID 2: 5103F3 Rabbit VH Amino Acid Sequence
QSVEESGGRLVTPGTPLTLTCTVSGIDLSGFAMGWVRQAPGKGPEYIGFIDSGGGAFYATWARG
RFTISRTSSTTVDLKMTSLTAADTATYFCARHGGNTYYYAMDPWGPGTLVTVSS
>Sequence ID 3: 5103F3 Rabbit VL Nucleic Acid Sequence
GCCCTTGTGATGACCCAGACTCCAGCCTCCGTGGAGGCAGCTGTGAGAGGCACAGTCACCATCA
AGTGCCAGGCCAGTCAGAGCATTAGTAACTACTTAGCCTGGTATCAGCAGAAACCAGGGCAGCC
TCCCAAGCTCCTGATCTATTCTGCATCCACTCTGGCATCTGGGGTCTCATCGCGGTTCAAAGGC
AGTGGATCTGGGACACAGTTCACTCTCACCATCAGCGGCGTGGAGTGTGCCGATGCTGCCACTT
ACTACTGTCAACAGGTGTATAGTGTCACTAATATTGATAATGCTTTCGGCGGAGGCACCGAGGT
GGAGTTCAAA
>Sequence ID 4: 5103F3 Rabbit VL Amino Acid Sequence
ALVMTQTPASVEAAVRGTVTIKCQASQSISNYLAWYQQKPGQPPKLLIYSASTLASGVSSRFKG
SGSGTQFTLTISGVECADAATYYCQQVYSVTNIDNAFGGGTEVEFK
>Sequence ID 5: 5103F3 BSM VH Nucleic Acid Sequence
CAGGTGCAGCTGCAGGAGTCCGGCGGCAGGCTGATCAAGCCCGGCGAGCCCCTGAGGCTGTCCT
GCAAGACCTCCGGCATCGACCTGTCCGGCTTCGCCATGGGCTGGGTGAGGCAGGCCCCCGGCAA
GGGCCTGGAGTACATCGGCTTCATCGACTCCGGCGGCGGCGCCTTCTACGCCACCTGGGCCAGG
GGCAGGTTCACCATCTCCAGGACCTCCACCAACACCGTGTACCTGCAGATGAACTCCCTGACCG
CCGAGGACACCGCCGTGTACTACTGCGCCAGGCACGGCGGCAACACCTACTACTACGCCATGGA
CCCCTGGGGCCCCGGCACCCTGGTGACCGTGTCCTCC
>Sequence ID 6: 5103F3 BSM VH Amino Acid Sequence
QVQLQESGGRLIKPGEPLRLSCKTSGIDLSGFAMGWVRQAPGKGLEYIGFIDSGGGAFYATWAR
GRFTISRTSTNTVYLQMNSLTAEDTAVYYCARHGGNTYYYAMDPWGPGTLVTVSS
>Sequence ID 7: 5103F3 BSM VL Nucleic Acid Sequence
GCCCTGAGGATGACCCAGTCCCCCTCCTCCCTGGCCGCCACCACCGGCCAGAGGGTGACCATCA
CCTGCCAGGCCTCCCAGTCCATCTCCAACTACCTGGCCTGGTACCAGCAGAAGCCCGGCCAGCC
CCCCAAGCTGCTGATCTACTCCGCCTCCACCCTGGCCTCCGGCGTGCCCTCCAGGTTCAAGGGC
TCCGGCTCCGGCACCCAGTTCACCCTGACCATCTCCAGCGTGCAGCCCGAGGACTTCGCCACCT
ACTACTGCCAGCAGGTGTACTCCGTGACCAACATCGACAACGCCTTCGGCGGCGGCACCAGGGT
GGAGATCAAG
>Sequence ID 8: 5103F3 BSM VL Amino Acid Sequence
ALRMTQSPSSLAATTGQRVTITCQASQSISNYLAWYQQKPGQPPKLLIYSASTLASGVPSRFKG
SGSGTQFTLTISCVQCEDFATYYCQQVYSVTNIDNAFGGGTRVEIK
>Sequence ID 9: 5103F3 FRS VH Nucleic Acid Sequence
CAGGTGCAGCTGGTGGAGTCCGGCGGCGGCCTGGTGAAGCCCGGCGGCTCCCTGAGGCTGTCCT
GCGCCGCCTCCGGCATCGACCTGTCCGGCTTCGCCATGGGCTGGGTGAGGCAGGCCCCCGGCAA
GGGCCTGGAGTGGGTGGGCTTCATCGACTCCGGCGGCGGCGCCTTCTACGCCACCTGGGCCAGG
GGCAGGTTCACCATCTCCAGGACCTCCAAGAACACCGTGTACCTGCAGATGAACTCCCTGAGGG
CCGAGGACACCGCCGTGTACTACTGCGCCAGGCACGGCGGCAACACCTACTACTACGCCATGGA
CCCCTGGGGCCAGGGCACCCTGGTGACCGTGTCCTCC
>Sequence ID 10: 5103F3 FRS VH Amino Acid Sequence
QVQLVESGGGLVKPGGSLRLSCAASGIDLSGFAMGWVRQAPGKGLEWVGFIDSGGGAFYATWAR
GRFTISRTSKNTVYLQMNSLRAEDTAVYYCARHGGNTYYYAMDPWGQGTLVTVSS
>Sequence ID 11: 5103F3 FRS VL Nucleic Acid Sequence
GCCCTGGTGATGACCCAGTCCCCCTCCTCCCTGTCCGCCTCCGTGGGCGACAGGGTGACCATCT
CCTGCCAGGCCTCCCAGTCCATCTCCAACTACCTGGCCTGGTACCAGCAGAAGCCCGGCCAGGC
CCCCAAGCTGCTGATCTACTCCGCCTCCACCCTGGCCTCCGGCGTGCCCTCCAGGTTCTCCGGC
TCCGGCTCCGGCACCGACTTCACCCTGACCATCTCCTCCGTGGAGCCCGAGGACTTCGCCACCT
ACTACTGCCAGCAGGTGTACTCCGTGACCAACATCGACAACGCCTTCGGCGGCGGCACCAAGGT
GGAGATCAAG
>Sequence ID 12: 5103F3 FRS VL Amino Acid Sequence
ALVMTQSPSSLSASVGDRVTISCQASQSISNYLAWYQQKPGQAPKLLIYSASTLASGVPSRFSG
SGSGTDFTLTISSVEPEDFATYYCQQVYSVTNIDNAFGGGTKVEIK
>Sequence ID 13: 5208H10 Rabbit VH Nucleic Acid Sequence
CAGTCGGTGAAGGAGTCCGAGGGAGGTCTCTTCAAGCCGACGGATACCCTGACACTCACCTGCA
CAGTCTCTGGATTCTCCCTCAGTACCTATGCAATGAGCTGGGTCCGCCAGGCTCCAGGGAACGG
GCTGGAATGGATCGGAATCATTGATGCTGTTGGTCGCACATATTACGCGAGTTGGGCGAAAAGT
CGATCCACCATCACCAGGAACACCAACCTGAACACGGTGACTCTGAAAATGACCAGTCTGACAG
CCGCGGACACGGCCACCTATTTCTGTGCGAGATGGTGGGATCTCTGGGGCCAAGGCACCCTGGT
CACCGTCTCGAGC
>Sequence ID 14: 5208H10 Rabbit VH Amino Acid Sequence
QSVKESEGGLFKPTDTLTLTCTVSGFSLSTYAMSWVRQAPGNGLEWIGIIDAVGRTYYASWAKS
RSTITRNTNLNTVTLKMTSLTAADTATYFCARWWDLWGQGTLVTVSS
>Sequence ID 15: 5208H10 Rabbit VL Nucleic Acid Sequence
GCGCAAGTGCTGACCCAGACTGCATCGTCCGTGTCTGCCGCTGTGGGAGGCACTGTCACCATCA
ATTGCCAGTCCAGTCAGAGTGTTTATGATAGCAACGACTTAACCTGGTATCAGCAGAAACCAGG
GCAGTCTCCCAAGCTCCTGATCTATCTGGCATCCACTCTGGCATCTGGGGTCCCATCGCGATTC
AAAGGCAGTGGATCTGGGACACAGTTCACTCTCACCATCAGCGACCTGGAGTGTGACGATTCTG
CCACTTACTACTGTGCAGCCCATTATAATAGTGATATTTATACTTTCGGCGGAGGGACCGAGGT
GGAGGTCAAA
>Sequence ID 16: 5208H10 Rabbit VL Amino Acid Sequence
AQVLTQTASSVSAAVGGTVTINCQSSQSVYDSNDLTWYQQKPGQSPKLLIYLASTLASGVPSRF
KGSGSGTQFTLTISDLECDDSATYYCAAHYNSDIYTFGGGTEVEVK
>Sequence ID 17: 5046A2 Rabbit VH Nucleic Acid Sequence
TTCTCTCCACAGGTGTCCACTCCCAGGTCCAAGTTTAAACGGATCTCTAGCGAATTCAAGCTTA
CGCTCACCATGGAGACTGGGCTGCGCTGGCTTCTCCTGGTCGCTGTGCTCAAAGGTGTCCAGTG
TCAGTCGGTGGAGGAGTCCGGGGGTCGCCTGATAACGCCTGGAGGATCCCTGACACTCACCTGC
ACCGTCTCTGGAATCGACCTCAGTAGCAATGGAATGAGCTGGGTCCGCCAGTCTCCAGGGAAGG
GGCTGGAATACATCGGATTCATTGATACTGATGGTAGCGCATACTCCGCGAGCTGGGCGAAAGG
CCGATTCACCATCTCCAAAGCCTCGTCGACCACGGTGGATCTGAAAATGACCAGTCTGACAACC
GAGGACACGGCCACCTATTTCTGTGCCAGAGCCTATGTTAGTGGTAGTACTGGTTACAATTTTA
ACATCTGGGGCCCGGGGACCCTGGTCACCGTCTCGAGCGCTAGCACCAAGGGCCCATCGGTCTT
CCCCCTGGCACCCTCCTCCAAGAGCACCTCTGGGGGCACAGCGGCCCTGGGCTGCT
>Sequence ID 18: 5046A2 Rabbit VH Amino Acid Sequence
FSPQVSTPRSKFKRISSEFKLTLTMETGLRWLLLVAVLKGVQCQSVEESGGRLITPGGSLTLTC
TVSGIDLSSNGMSWVRQSPGKGLEYIGFIDTDGSAYSASWAKGRFTISKASSTTVDLKMTSLTT
EDTATYFCARAYVSGSTGYNFNIWGPGTLVTVSSASTKGPSVFPLAPSSKSTSGGTAALGC
>Sequence ID 19: 5046A2 Rabbit VL Nucleic Acid Sequence
TTCTCTCCACAGGTGTCCACTCCCAGGTCCAAGTTTAAACGGATCTCTAGCGAATTCAAGCTTC
GAATCGACATGGACACGAGGGCCCCCACTCAGCTGCTGGGGCTCCTGCTGCTCTGGCTCCCAGG
TGCCAGATGTGCATTCGAATTGACCCAGACTCCATCCTCCGTGGAGGCAGCTGTGGGAGGCACA
GTCACCATCAAGTGCCAGGCCAGTCAGAGCATTAGTAGCTACTTAGCCTGGTATCAGCAGAAAC
CAGGGCAGCCTCCCAAGTCCCTGATCTACAAGGCATCCACTCTGGCATCTGGGGTCCCATCGCG
GTTCAAAGGCAGTGGATCTGGGACAGAGTTCACTCTCACCATCAGCGACCTGGAGTGTGCCGAT
GCTGCCACTTACTACTGTCAACAGGGTTATACTATTCGTAATATTGATAATGCTTTCGGCGGAG
GGACCGAGGTGGAGTTCAAACGTACGGTGGCTGCACCATCTGTCTTCATCTTCCCGCCATCTGA
TGAGCAGTTGAAATCTGGAACTGCCTCT
>Sequence ID 20: 5046A2 Rabbit VL Amino Acid Sequence
FSPQVSTPRSKFKRISSEFKLRIDMDTRAPTQLLGLLLLWLPGARCAFELTQTPSSVEAAVGGT
VTIKCQASQSISSYLAWYQQKPGQPPKSLIYKASTLASGVPSRFKGSGSGTEFTLTISDLECAD
AATYYCQQGYTIRNIDNAFGGGTEVEFKRTVAAPSVFIFPPSDEQLKSGTAS
>Sequence ID 21: 5046A2 BSM VH Nucleic Acid Sequence
CAGGTGCAGCTGGTGGAGTCCGGCGGCAGGCTGATCAAGCCCGGCGGCTCCCTGAGGCTGACCT
GCAAGACCTCCGGCATCGACCTGTCCTCCAACGGCATGTCCTGGGTGAGGCAGCCCCCCGGCAA
GGGCCTGGAGTACATCGGCTTCATCGACACCGACGGCTCCGCCTACTCCGCCTCCTGGGCCAAG
GGCAGGTTCACCATCTCCAAGGCCTCCATCAACACCGTGTTCCTGCAGATGACCTCCCTGAAGT
CCGAGGACACCGCCATCTACTACTGCGCCAGGGCCTACGTGTCCGGCTCCACCGGCTACAACTT
CAACATCTGGGGCCCCGGCACCCTGGTGACCGTGTCCTCC
>Sequence ID 22: 5046A2 BSM VH Amino Acid Sequence
QVQLVESGGRLIKPGGSLRLTCKTSGIDLSSNGMSWVRQPPGKGLEYIGFIDTDGSAYSASWAK
GRFTISKASINTVFLQMTSLKSEDTAIYYCARAYVSGSTGYNFNIWGPGTLVTVSS
>Sequence ID 23: 5046A2 BSM VL Nucleic Acid Sequence
GCCTTCAGGCTGACCCAGACCCCCTCCTCCTTCGCCGCCACCCTGGGCCAGAGGGTGACCATCA
CCTGCCAGGCCTCCCAGTCCATCTCCTCCTACCTGGCCTGGTACCAGCAGAAGCCCGGCAAGCC
CCCCAAGTCCCTGATCTACAAGGCCTCCACCCTGGCCTCCGGCGTGCCCTCCAGGTTCAAGGGC
TCCGGCTCCGGCACCGACTTCACCCTGACCATCTCCAGCCTGCAGCCCGACGACTTCGCCACCT
ACTACTGCCAGCAGGGCTACACCATCAGGAACATCGACAACGCCTTCGGCGGCGGCACCAGGGT
GGAGATCAAG
>Sequence ID 24: 5046A2 BSM VL Amino Acid Sequence
AFRLTQTPSSFAATLGQRVTITCQASQSISSYLAWYQQKPGKPPKSLIYKASTLASGVPSREKG
SGSGTDFTLTISSLQPDDFATYYCQQGYTIRNIDNAFGGGTRVEIK
>Sequence ID 25: 5046A2 FRS VH Nucleic Acid Sequence
CAGGTGCAGCTGGTGGAGTCCGGCGGCGGCCTGATCAAGCCCGGCGGCTCCCTGAGGCTGTCCT
GCGCCGCCTCCGGCATCGACCTGTCCTCCAACGGCATGTCCTGGGTGAGGCAGGCCCCCGGCAA
GGGCCTGGAGTGGGTGGGCTTCATCGACACCGACGGCTCCGCCTACTCCGCCTCCTGGGCCAAG
GGCAGGTTCACCATCTCCAAGGACTCCAAGAACACCGTGTACCTGCAGATGAACTCCCTGAGGG
CCGAGGACACCGCCGTGTACTACTGCGCCAGGGCCTACGTGTCCGGCTCCACCGGCTACAACTT
CAACATCTGGGGCCAGGGCACCCTGGTGACCGTGTCCTCC
>Sequence ID 26: 5046A2 FRS VH Amino Acid Sequence
QVQLVESGGGLIKPGGSLRLSCAASGIDLSSNGMSWVRQAPGKGLEWVGFIDTDGSAYSASWAK
GRFTISKDSKNTVYLQMNSLRAEDTAVYYCARAYVSGSTGYNFNIWGQGTLVTVSS
>Sequence ID 27: 5046A2 FRS VL Nucleic Acid Sequence
GCCTTCGTGCTGACCCAGTCCCCCTCCTCCCTGTCCGCCTCCGTGGGCGACAGGGTGACCATCT
CCTGCCAGGCCTCCCAGTCCATCTCCTCCTACCTGGCCTGGTACCAGCAGAAGCCCGGCCAGGC
CCCCAAGTCCCTGATCTACAAGGCCTCCACCCTGGCCTCCGGCGTGCCCTCCAGGTTCTCCGGC
TCCGGCTCCGGCACCGACTTCACCCTGACCATCTCCTCCCTGGAGCCCGAGGACTTCGCCACCT
ACTACTGCCAGCAGGGCTACACCATCAGGAACATCGACAACGCCTTCGGCGGCGGCACCAAGGT
GGAGATCAAG
>Sequence ID 28: 5046A2 FRS VL Amino Acid Sequence
AFVLTQSPSSLSASVGDRVTISCQASQSISSYLAWYQQKPGQAPKSLIYKASTLASGVPSRFSG
SGSGTDFTLTISSLEPEDFATYYCQQGYTIRNIDNAFGGGTKVEIK
>Sequence ID 29: 5111D12 Rabbit VH Nucleic Acid Sequence
TTCTCTCCACAGGTGTCCACTCCCAGGTCCAAGTTTAAACGGATCTCTAGCGAATTCAAGCTTA
CGCTCACCATGGAGACTGGGCTGCGCTGGCTTCTCCTGNNCGCTGTGCTCAAAGGTGTCCAGTG
TCAGTCGGTGGAGGAGTCCGGGGGTCGCCTGGTCACGCCTGGGACAGCCCTGACACTCACCTGC
ACAGTCTCTGGATTCTCCCTCAGTAGCTATTCAATGGGCTGGGTCCGCCAGGCTCCAGGGAAGG
GGCTGGAGTGGATCGGATATCTTAATACTGGTGGTAGCACATACTACGCGAGCTGGGTGAATGG
TCGATTCACCATCTCCAAAACCTCGTCGACCACGGTGGATCTGAAAATCACCAGTCCGACAACC
GAGGACACGGCCACCTATTTCTGTACCAGGGCCGGGGGTGTTAGTAGCAATATGGGCTTTAACA
TCTGGGGCCAAGGGACCCTGGTCACCGTCTCGAGCGCTAGCACCAAGGNNCCATCGGTCTTCCC
CCTGGCACCCTCCTCCAAGAGCACCTCTGGGGGCACAGCGGCCCTGGGC
>Sequence ID 30: 5111D12 Rabbit VH Amino Acid Sequence
FSPQVSTPRSKFKRISSEFKLTLTMETGLRWLLLXAVLKGVQCQSVEESGGRLVTPGTALTLTC
TVSGFSLSSYSMGWVRQAPGKGLEWIGYLNTGGSTYYASWVNGRFTISKTSSTTVDLKITSPTT
EDTATYFCTRAGGVSSNMGFNIWGQGTLVTVSSASTKXPSVFPLAPSSKSTSGGTAALG
>Sequence ID 31: 5111D12 Rabbit VL Nucleic Acid Sequence
TTCTCTCCACAGGTGTCCACTCCCAGGTCCAAGTTTAAACGGATCTCTAGCGAATTCAAGCTTC
GAATCGACATGGACACGAGGGCCCCCACTCAGCTGCTGGGGCTCCTGCTGCTCTGGCTCCCAGG
TGCCAGATGTGACCCTGTGCTGACCCAGACTCCAGCCTCCGTGTCTGAGCCTGTGGGAGGCACA
GTCACCATCAAGTGCCAGGCCAGTCAGAGCATTAGTAGCTACTTAGCCTGGTATCAGCAGAAAC
CAGGGCAGCCTCCCAAGCTCCTGATCTACAGGGCATCCACTCTGGCATCTGGGGTCCCATCGCG
GTTCAAAGGCAGTGGATCTGGGACACAGTTCACTCTCACCATCAGCGACCTGGAGTGTGCCGAT
GCTGCCACTTACTACTGTCAATGTACTGATTATGGCTACAATTATCTTGGGGCTTTCGGCGGAG
GGACCGAGGTGGAGTTCAAACGTACGGTGGCTGCACCATCTGTCTTCATCTTCCCGCCATCTGA
TGAGCAGTTGAAATCTGGAACTGCCTCTG
>Sequence ID 32: 5111D12 Rabbit VL Amino Acid Sequence
FSPQVSTPRSKFKRISSEFKLRIDMDTRAPTQLLGLLLLWLPGARCDPVLTQTPASVSEPVGGT
VTIKCQASQSISSYLAWYQQKPGQPPKLLIYRASTLASGVPSRFKGSGSGTQFTLTISDLECAD
AATYYCQCTDYGYNYLGAFGGGTEVEFKRTVAAPSVFIFPPSDEQLKSGTAS
>Sequence ID 33: 5111D12 BSM VH Nucleic Acid Sequence
CAGGTGCAGCTGCTGGAGTCCGGCGGCAGGCTGATCAAGCCCGGCGAGTCCCTGAAGCTGACCT
GCAAGACCTCCGGCTTCTCCCTGTCCTCCTACTCCATGGGCTGGGTGAGGCAGGCCCCCGGCAA
GGGCCTGGAGTGGATCGGCTACCTGAACACCGGCGGCTCCACCTACTACGCCTCCTGGGTGAAC
GGCAGGTTCACCATCTCCAAGACCTCCATCTCCACCGTGTTCCTGCAGCTGAGGTCCCCCAGGT
CCGAGGACACCGCCCTGTACTACTGCACCAGGGCCGGCGGCGTGTCCTCCAACATGGGCTTCAA
CATCTGGGGCCAGGGCACCCTGGTGACCGTGTCCTCC
>Sequence ID 34: 5111D12 BSM VH Amino Acid Sequence
QVQLLESGGRLIKPGESLKLTCKTSGFSLSSYSMGWVRQAPGKGLEWIGYLNTGGSTYYASWVN
GRFTISKTSISTVFLQLRSPRSEDTALYYCTRAGGVSSNMGFNIWGQGTLVTVSS
>Sequence ID 35: 5111D12 BSM VL Nucleic Acid Sequence
GACCCCGTGCTGACCCAGTCCCCCTCCTTCCTGTCCCTGACCGTGGGCCAGAGGGTGACCATCA
ACTGCCAGGCCTCCCAGTCCATCTCCTCCTACCTGGCCTGGTACCAGCAGAAGCCCGGCCAGCC
CCCCAAGCTGCTGATCTACAGGGCCTCCACCCTGGCCTCCGGCGTGCCCTCCAGGTTCTCCGGC
TCCGGCTCCGGCACCGAGTTCACCCTGACCATCTCCTCCCTGCAGTCCGACGACTTCGCCACCT
ACTACTGCCAGTGCACCGACTACGGCTACAACTACCTGGGCGCCTTCGGCGGCGGCACCAGGGT
GGAGATCAAG
>Sequence ID 36: 5111D12 BSM VL Amino Acid Sequence
DPVLTQSPSFLSLTVGQRVTINCQASQSISSYLAWYQQKPGQPPKLLIYRASTLASGVPSRFSG
SGSGTEFTLTISSLQSDDFATYYCQCTDYGYNYLGAFGGGTRVEIK
>Sequence ID 37: 5194G6 Rabbit VH Nucleic Acid Sequence
CAGGAGCAGCTGAAGGAGTCCGGGGGAGGCCTGGTCCAGCCTGGGGGATCCCTGACACTCACCT
GCAAAGCCTCTGGATTCTCCTTCAGTAGCGTCTACTACATGTGCTGGGTCCGCCAGGCTCCAGG
GAAGGGGCTGGAGTGGATCGGATGCAGTTATACGAGTGATGGTAGCGCTTACTACGCGAACTGG
GCGACAGGCCGATTCACCATCTCCAAAACCTCGTCGACCACGGTGACTCTGCAAATGACGAGTC
TGACAGACGCGGACACGGCCACCTATTTCTGTGCGAGAGATAGGACTGATAGTCGTGATTATTT
TTTTGACTTGTGGGGCCCAGGCACCCTGGTCACCGTCTCGAGC
>Sequence ID 38: 5194G6 Rabbit VH Amino Acid Sequence
QEQLKESGGGLVQPGGSLTLTCKASGFSFSSVYYMCWVRQAPGKGLEWIGCSYTSDGSAYYANW
ATGRFTISKTSSTTVTLQMTSLTDADTATYFCARDRTDSRDYFFDLWGPGTLVTVSS
>Sequence ID 39: 5194G6 Rabbit VL Nucleic Acid Sequence
GCCTATGATATGACCCAGACTCCAGCCTCCGTGGAGGCAGCTGTGGGAGGCACAGTCAGCATCA
GTTGTCAGTCCAGTCAGAATGTTTATAGTAACTACTTATCCTGGTATCAGCAGAAACCAGGGCA
GCCTCCCAAGCTCCTGATCTACAGGGCATCCACTCTGGCTTCTGGGGTCTCATCGCGATTCAGA
GGCAGTGGATCCGGGACACAGTTCACTCTCACCATCAGCGGCGTGGAGTGTGCCGATGCTGCCA
CTTACTATTGTCAACAGGGTTATAAATATGGTAATGTTAATAATCTTTTCGGCGGAGGGACCGA
GGTGGAGGTCAAA
>Sequence ID 40: 5194G6 Rabbit VL Amino Acid Sequence
AYDMTQTPASVEAAVGGTVSISCQSSQNVYSNYLSWYQQKPGQPPKLLIYRASTLASGVSSRFR
GSGSGTQFTLTISGVECADAATYYCQQGYKYGNVNNLFGGGTEVEVK
>Sequence ID 41: 5194G6 BSM VH Nucleic Acid Sequence
GAGGAGCAGCTGCTGGAGTCCGGCGGCGGCCTGGTGCAGCCCGGCGGCTCCCTGAGGCTGTCCT
GCACCGCCTCCGGCTTCACCTTCGAGTCCGTGTACTACATGTGCTGGGTGAGGCAGGCCCCCGG
CAAGGGCCTGGAGTGGATCGGCTGCTCCTACACCTCCGACGGCTCCGCCTACTACGCCAACTGG
GCCACCGGCAGGTTCACCATCTCCAAGACCTCCATCAACACCGTGTTCCTGCAGATGAGGTCCC
TGAGGTCCGAGGACACCGCCATCTACTACTGCGCCAGGGACAGGACCGACTCCAGGGACTACTT
CTTCGACCTGTGGGGCCCCGGCACCCTGGTGACCGTGTCCTCC
>Sequence ID 42: 5194G6 BSM VH Amino Acid Sequence
EEQLLESGGGLVQPGGSLRLSCTASGFTFESVYYMCWVRQAPGKGLEWIGCSYTSDGSAYYANW
ATGRFTISKTSINTVFLQMRSLRSEDTAIYYCARDRTDSRDYFFDLWGPGTLVTVSS
>Sequence ID 43: 5194G6 BSM VL Nucleic Acid Sequence
GCCTACAGGATGACCCAGTCCCCCTCCTCCTTCTCCGCCTCCACCGGCCAGAGGGTGACCATCA
CCTGCCAGTCCTCCCAGAACGTGTACTCCAACTACCTGTCCTGGTACCAGCAGAAGCCCGGCAA
GCCCCCCAAGCTGCTGATCTACAGGGCCTCCACCCTGGCCTCCGGCGTGCCCTCCAGGTTCAGG
GGCTCCGGCTCCGGCACCCAGTTCACCCTGACCATCTCCTGCGTGCAGTGCGACGACTTCGCCA
CCTACTACTGCCAGCAGGGCTACAAGTACGGCAACGTGAACAACCTGTTCGGCGGCGGCACCAG
GGTGGAGATCAAG
>Sequence ID 44: 5194G6 BSM VL Amino Acid Sequence
AYRMTQSPSSFSASTGQRVTITCQSSQNVYSNYLSWYQQKPGKPPKLLIYRASTLASGVPSRFR
GSGSGTQFTLTISCVQCDDFATYYCQQGYKYGNVNNLFGGGTRVEIK
>Sequence ID 45: 5194G6 FRS VH Nucleic Acid Sequence
CAGGAGCAGCTGGTGGAGTCCGGCGGCGGCCTGGTGCAGCCCGGCGGCTCCCTGAGGCTGTCCT
GCGCCGCCTCCGGCTTCACCTTCTCCTCCGTGTACTACATGTGCTGGGTGAGGCAGGCCCCCGG
CAAGGGCCTGGAGTGGGTGGGCTGCTCCTACACCTCCGACGGCTCCGCCTACTACGCCAACTGG
GCCACCGGCAGGTTCACCATCTCCAAGACCTCCAAGAACACCGTGTACCTGCAGATGAACTCCC
TGAGGGCCGAGGACACCGCCGTGTACTACTGCGCCAGGGACAGGACCGACTCCAGGGACTACTT
CTTCGACCTGTGGGGCCAGGGCACCCTGGTGACCGTGTCCTCC
>Sequence ID 46: 5194G6 FRS VH Amino Acid Sequence
QEQLVESGGGLVQPGGSLRLSCAASGFTFSSVYYMCWVRQAPGKGLEWVGCSYTSDGSAYYANW
ATGRFTISKTSKNTVYLQMNSLRAEDTAVYYCARDRTDSRDYFFDLWGQGTLVTVSS
>Sequence ID 47: 5194G6 FRS VL Nucleic Acid Sequence
GCCTACGTGATGACCCAGTCCCCCTCCTCCCTGTCCGCCTCCGTGGGCGACAGGGTGACCATCT
CCTGCCAGTCCTCCCAGAACGTGTACTCCAACTACCTGTCCTGGTACCAGCAGAAGCCCGGCCA
GGCCCCCAAGCTGCTGATCTACAGGGCCTCCACCCTGGCCTCCGGCGTGCCCTCCAGGTTCTCC
GGCTCCGGCTCCGGCACCGACTTCACCCTGACCATCTCCTCCGTGGAGCCCGAGGACTTCGCCA
CCTACTACTGCCAGCAGGGCTACAAGTACGGCAACGTGAACAACCTGTTCGGCGGCGGCACCAA
GGTGGAGATCAAG
>Sequence ID 48: 5194G6 FRS VL Amino Acid Sequence
AYVMTQSPSSLSASVGDRVTISCQSSQNVYSNYLSWYQQKPGQAPKLLIYRASTLASGVPSRFS
GSGSGTDFTLTISSVEPEDFATYYCQQGYKYGNVNNLFGGGTKVEIK
>Sequence ID 49: 5194G6 GS VH Nucleic Acid Sequence
CAGGAGCAGCTGCTGGAGTCCGGCGGCGGCCTGGTGCAGCCCGGCGGCTCCCTGAGGCTGTCCT
GCGCCGCCTCCGGCTTCACCTTCTCCTCCGTGTACTACATGTGCTGGGTGAGGCAGGCCCCCGG
CAAGGGCCTGGAGTGGGTGGGCTGCTCCTACACCTCCGACGGCTCCGCCTACTACGCCAACTGG
GCCACCGGCAGGTTCACCATCTCCAAGACCTCCAAGAACACCGTGTACCTGCAGATGAACTCCC
TGAGGGCCGAGGACACCGCCGTGTACTACTGCGCCAGGGACAGGACCGACTCCAGGGACTACTT
CTTCGACCTGTGGGGCCAGGGCACCCTGGTGACCGTGTCCTCC
>Sequence ID 50: 5194G6 GS VH Amino Acid Sequence
QEQLLESGGGLVQPGGSLRLSCAASGFTFSSVYYMCWVRQAPGKGLEWVGCSYTSDGSAYYANW
ATGRFTISKTSKNTVYLQMNSLRAEDTAVYYCARDRTDSRDYFFDLWGQGTLVTVSS
>Sequence ID 51: 5194G6 GS VL Nucleic Acid Sequence
GCCTACAGGATGACCCAGTCCCCCTCCTCCGTGTCCGCCTCCGTGGGCGACAGGGTGACCATCT
CCTGCCAGTCCTCCCAGAACGTGTACTCCAACTACCTGTCCTGGTACCAGCAGAAGCCCGGCCA
GGCCCCCAAGCTGCTGATCTACAGGGCCTCCACCCTGGCCTCCGGCGTGCCCTCCAGGTTCTCC
GGCTCCGGCTCCGGCACCGACTTCACCCTGACCATCTCCGGCGTGCAGTCCGAGGACTTCGCCA
CCTACTACTGCCAGCAGGGCTACAAGTACGGCAACGTGAACAACCTGTTCGGCGGCGGCACCAA
GGTGGAGATCAAG
>Sequence ID 52: 5194G6 GS VL Amino Acid Sequence
AYRMTQSPSSVSASVGDRVTISCQSSQNVYSNYLSWYQQKPGQAPKLLIYRASTLASGVPSRFS
GSGSGTDFTLTISGVQSEDFATYYCQQGYKYGNVNNLFGGGTKVEIK
>Sequence ID 53: 5119H11 Rabbit VH Nucleic Acid Sequence
CAGTCGGTGGAGGAGTCCGGGGGTCGCCTGGTCACGCCTGGGACACCCCTGACACTCACCTGCA
CGGTCTCTGGATTCTCCCTCAGTACCTATTCAATGGGCTGGGTCCGCCAGGCTCCAGGGAAGGG
GCTGGAATGGATCGGAGTCATTTACTATGGTGAGCCCACATACTGCGCGGCCTGGGCGAAAGGC
CGATTCACCATCTCCAAAACCTCGTCGACCACGGTGGATCTGAAAATCACCAGTCCGACAACCG
AGGACACGGCCACCTATTTCTGTGCCAGATCAGGTGATCGTAATGGCTACTACTTTAACATCTG
GGGCCCAGGCACCCTGGTCACCGTCTCGAGC
>Sequence ID 54: 5119H11 Rabbit VH Amino Acid Sequence
QSVEESGGRLVTPGTPLTLTCTVSGFSLSTYSMGWVRQAPGKGLEWIGVIYYGEPTYCAAWAKG
RFTISKTSSTTVDLKITSPTTEDTATYFCARSGDRNGYYFNIWGPGTLVTVSS
>Sequence ID 55: 5119H11 Rabbit VL Nucleic Acid Sequence
GCCTATGATATGACCCAGACTCCAGCCTCCGTGTCTGCAGCTGTGGGAGGCACAGTCACCATCA
AGTGCCAGGCCAGTCAGAGCATTAGTAGCTACTTAGCCTGGTATCAGCAGAAACCAGGGCAGCC
TCCCAAGCTCCTGATCTATTATGCATCCACTCTGGCATCTGGGGTCCCATCGCGGTTCAAAGGC
AGTGGATCTGGGACAGAGTTCACTCTCACCATCAGCGGCGTGCAGTGTGCCGATGCAGCCACTT
ACTACTGTCAACAGGTTGCTGTTATTGGTAATGTTGGGGATAATACTTTCGGCGGAGGGACCGA
GGTGGAGGTCAAA
>Sequence ID 56: 5119H11 Rabbit VL Amino Acid Sequence
AYDMTQTPASVSAAVGGTVTIKCQASQSISSYLAWYQQKPGQPPKLLIYYASTLASGVPSRFKG
SGSGTEFTLTISGVQCADAATYYCQQVAVIGNVGDNTFGGGTEVEVK
>Sequence ID 57: 5103F3 VH-CDRH1
GFAMG
>Sequence ID 58: 5103F3 VH-CDRH2
FIDSGGGAFYATWARG
>Sequence ID 59: 5103F3 VH-CDRH3
HGGNTYYYAMDP
>Sequence ID 60: 5103F3 VL-CDRL1
QASQSISNYLA
>Sequence ID 61: 5103F3 VL-CDRL2
SASTLAS
>Sequence ID 62: 5103F3 VL-CDRL3
QQVYSVTNIDNA
>Sequence ID 63: 5208H10 VH-CDRH1
TYAMS
>Sequence ID 64: 5208H10 VH-CDRH2
IIDAVGRTYYASWAKS
>Sequence ID 65: 5208H10 VH-CDRH3
WWDL
>Sequence ID 66: 5208H10 VL-CDRL1
QSSQSVYDSNDLT
>Sequence ID 67: 5208H10 VL-CDRL2
LASTLASGVPS
>Sequence ID 68: 5208H10 VL-CDRL3
AAHYNSDIYT
>Sequence ID 69: 5046A2 VH-CDRH1
SNGMS
>Sequence ID 70: 5046A2 VH-CDRH2
FIDTDGSAYSASWAKG
>Sequence ID 71: 5046A2 VH-CDRH3
AYVSGSTGYNfNI
>Sequence ID 72: 5046A2 VL-CDRL1
QASQSISSYLA
>Sequence ID 73: 5046A2 VL-CDRL2
KASTLAS
>Sequence ID 74: 5046A2 VL-CDRL3
QQGYTIRNIDNA
>Sequence ID 75: 5111D12 VH-CDRH1
SYSMG
>Sequence ID 76: 5111D12 VH-CDRH2
YLNTGGSTYYASWVNG
>Sequence ID 77: 5111D12 VH-CDRH3
AGGVSSNMGFNI
>Sequence ID 78: 5111D12 VL-CDRL1
QASQSISSYLA
>Sequence ID 79: 5111D12 VL-CDRL2
RASTLAS
>Sequence ID 80: 5111D12 VL-CDRL3
QCTDYGYNYLGA
>Sequence ID 81: 5119H11 VH-CDRH1
TYSMG
>Sequence ID 82: 5119H11 VH-CDRH2
VIYYGEPTYCAAWAKG
>Sequence ID 83: 5119H11 VH-CDRH3
SGDRNGYYFNI
> Sequence ID 84: 5119H11 VL-CDRL1
QASQSISSYLA
>Sequence ID 85: 5119H11 VL-CDRL2
YASTLAS
>Sequence ID 86: 5119H11 VL-CDRL3
QQVAVIGNVGDNT
>Sequence ID 87: Human Claudin18.2 Nucleic Acid Sequence
ATGGCCGTGACCGCCTGCCAGGGCCTGGGCTTCGTGGTGAGCCTGATCGGCATCGCCGGCATCA
TCGCCGCCACCTGCATGGACCAGTGGAGCACCCAGGACCTGTACAACAACCCCGTGACCGCCGT
GTTCAACTACCAGGGCCTGTGGAGGAGCTGCGTGAGGGAGAGCAGCGGCTTCACCGAGTGCAGG
GGCTACTTCACCCTGCTGGGCCTGCCCGCCATGCTGCAGGCCGTGAGGGCCCTGATGATCGTGG
GCATCGTGCTGGGCGCCATCGGCCTGCTGGTGAGCATCTTCGCCCTGAAGTGCATCAGGATCGG
CAGCATGGAGGACAGCGCCAAGGCCAACATGACCCTGACCAGCGGCATCATGTTCATCGTGAGC
GGCCTGTGCGCCATCGCCGGCGTGAGCGTGTTCGCCAACATGCTGGTGACCAACTTCTGGATGA
GCACCGCCAACATGTACACCGGCATGGGCGGCATGGTGCAGACCGTGCAGACCAGGTACACCTT
CGGCGCCGCCCTGTTCGTGGGCTGGGTGGCCGGCGGCCTGACCCTGATCGGCGGCGTGATGATG
TGCATCGCCTGCAGGGGCCTGGCCCCCGAGGAGACCAACTACAAGGCCGTGAGCTACCACGCCA
GCGGCCACAGCGTGGCCTACAAGCCCGGCGGCTTCAAGGCCAGCACCGGCTTCGGCAGCAACAC
CAAGAACAAGAAGATCTACGACGGCGGCGCCAGGACCGAGGACGAGGTGCAGAGCTACCCCAGC
AAGCACGACTACGTGTGA
>Sequence ID 88: Human Claudin18.1 Nucleic Acid Sequence
ATGTCCACCACCACATGCCAAGTGGTGGCGTTCCTCCTGTCCATCCTGGGGCTGGCCGGCTGCA
TCGCGGCCACCGGGATGGACATGTGGAGCACCCAGGACCTGTACGACAACCCCGTCACCTCCGT
GTTCCAGTACGAAGGGCTCTGGAGGAGCTGCGTGAGGCAGAGTTCAGGCTTCACCGAATGCAGG
CCCTATTTCACCATCCTGGGACTTCCAGCCATGCTGCAGGCAGTGCGAGCCCTGATGATCGTAG
GCATCGTCCTGGGTGCCATTGGCCTCCTGGTATCCATCTTTGCCCTGAAATGCATCCGCATTGG
CAGCATGGAGGACTCTGCCAAAGCCAACATGACACTGACCTCCGGGATCATGTTCATTGTCTCA
GGTCTTTGTGCAATTGCTGGAGTGTCTGTGTTTGCCAACATGCTGGTGACTAACTTCTGGATGT
CCACAGCTAACATGTACACCGGCATGGGTGGGATGGTGCAGACTGTTCAGACCAGGTACACATT
TGGTGCGGCTCTGTTCGTGGGCTGGGTCGCTGGAGGCCTCACACTAATTGGGGGTGTGATGATG
TGCATCGCCTGCCGGGGCCTGGCACCAGAAGAAACCAACTACAAAGCCGTTTCTTATCATGCCT
CAGGCCACAGTGTTGCCTACAAGCCTGGAGGCTTCAAGGCCAGCACTGGCTTTGGGTCCAACAC
CAAAAACAAGAAGATATACGATGGAGGTGCCCGCACAGAGGACGAGGTACAATCTTATCCTTCC
AAGCACGACTATGTGTAA

What is claimed is:

1. An isolated monoclonal antibody (mAb) or antigen-binding fragment thereof having a binding specificity to CLDN18, comprising an amino acid sequence having at least 90% identity with the SEQ ID NO:2, 4, 6, 8, 10, 12, 14, 16, 18, 20, 22, 24, 26, 28, 30, 32, 34, 36, 38, 40, 42, 44, 46, 48, 50, 52, 54, or 56.

2. The isolated mAb or antigen-binding fragment of claim 1, wherein the isolated mAb or antigen-binding fragment thereof selectively binds to CLDN18.2 but not CLDN18.1.

3. The isolated mAb or antigen-binding fragment thereof of claim 1, further comprising a human framework region.

4. The isolated mAb or antigen-binding fragment thereof of claim 1, wherein the isolated mAb is a humanized antibody, a chimeric antibody, or a recombinant antibody.

5. The isolated mAb or antigen-binding fragment thereof of claim 1, wherein the isolated mAb is an IgG.

6. The isolated mAb or antigen-binding fragment thereof of claim 1, wherein the antigen-binding fragment is a Fv, a Fab, a F(ab′)2, a scFv, or a scFv2 fragment.

7. The isolated mAb or antigen-binding fragment thereof of claim 1, wherein the isolated mAb is a bispecific antibody, tri-specific antibody, or multi-specific antibody.

8. The isolated mAb or antigen-binding fragment thereof of claim 1, comprising an IgG1 heavy chain having an amino acid sequence having at least 90% identity with the SEQ ID NO: 2, 6, 10, 14, 18, 22, 26, 30, 34, 38, 42, 46, 50, or 54.

9. The isolated mAb or antigen-binding fragment thereof of claim 1, comprising a kappa light chain having an amino acid sequence having at least 90% identity the SEQ ID NO: 4, 8, 12, 16, 20, 24, 28, 32, 36, 40, 44, 48, 52, or 56.

10. The isolated mAb or antigen-binding fragment thereof of claim 1, comprising a variable heavy chain having an amino acid sequence having at least 90% identity with the SEQ ID NO: 2, 6, 10, 14, 18, 22, 26, 30, 34, 38, 42, 46, 50, or 54, or a variable light chain having an amino acid sequence having at least 90% identity with the SEQ ID NO: 4, 8, 12, 16, 20, 24, 28, 32, 36, 40, 44, 48, 52, or 56.

11. An isolated mAb or antigen-binding fragment thereof having a binding specificity to CLDN18, wherein the isolated mAb or antigen-binding fragment thereof comprises a variable heavy chain having complementarity determining region (CDR) 1, CDR2, and CDR3 of SEQ ID NO: 57, 58, and 59; 63, 64, and 65; 69, 70, and 71; 75, 76, and 77; or 81, 82, and 83; and a variable light chain having CDR 1, CDR2, and CDR3 of SEQ ID NO:60, 61, and 62; 66, 67, and 68; 72, 73, and 74; 78, 79, and 80; or 84, 85, and 86.

12. An isolated nucleic acid encoding the isolated mAb or antigen-binding fragment of claim 1, the IgG1 heavy Chain of claim 8, the kappa light chain of claim 9, the variable light chain of claim 10, or the variable heavy chain of claim 11.

13. An expression vector comprising the isolated nucleic acid of claim 12, wherein the vector is expressible in a cell.

14. A host cell comprising the nucleic acid of claim 12, wherein the host cell is a prokaryotic cell or a eukaryotic cell.

15. A method of producing an antibody comprising culturing the host cell of one of claim 14, so that the antibody is produced.

16. An immuno-conjugate, comprising the isolated mAb or antigen-binding fragment thereof of claim 1 conjugated to a drug unit through a linker, wherein the linker comprises a covalent bond selected from an ester bond, an ether bond, an amine bond, an amide bond, a disulfide bond, an imide bond, a sulfone bond, a phosphate bond, a phosphorus ester bond, a peptide bond, a hydrazone bond or a combination thereof.

17. The immuno-conjugate according to claim 16, wherein the drug unit comprises a cytotoxic agent, an immune regulatory reagent, or a combination thereof.

18. The immuno-conjugate according to claim 16, wherein the cytotoxic agent is selected from a growth inhibitory agent or a chemotherapeutic agent from a class of tubulin binders, DNA intercalators, DNA alkylators, enzyme inhibitors, immune modulators, antimetabolite agents, radioactive isotopes, or a combination thereof.

19. The immuno-conjugate according to claim 16, wherein the cytotoxic agent is selected from a calicheamicin, ozogamicin, monomethyl auristatin E, emtansinea derivative or a combination thereof.

20. The immuno-conjugate according to claim 16, wherein the immune regulatory reagents activate or suppress immune cells, T cell, NK cell, B cell, macrophage, or dendritic cell.

21. A pharmaceutical composition, comprising the isolated mAb or antigen-binding fragment thereof of claim 1 or the immuno-conjugate of claim 19, and a pharmaceutically acceptable carrier.

22. The pharmaceutical composition of claim 21, further comprising a radioisotope, a radionuclide, a toxin, a therapeutic agent, a chemotherapeutic agent or a combination thereof.

23. The pharmaceutical composition of claim 22, wherein the therapeutic agent comprises an anti-estrogen agent, a receptor tyrosine kinase inhibitor, a kinase inhibitor, a cell cycle inhibitor, a DNA, RNA or protein synthesis inhibitor, a RAS inhibitor, or a combination thereof.

24. A method of treating a subject with a cancer, comprising administering to the subject an effective amount of the isolated mAb or antigen-binding fragment thereof of claim 1.

25. The method of claim 24, wherein the cancer comprises cells expressing Claudin 18.2.

26. The method of claim 24, wherein the cancer comprises colorectal cancer, pancreatic cancer, esophageal cancer, nasopharyngeal cancer, anal cancer, rectal cancer, gastric cancer, or bladder cancer.

27. The method of claim 24, further comprising co-administering an effective amount of a therapeutic agent.

28. The method of claim 24, wherein the therapeutic agent comprises an antibody, a chemotherapy agent, an enzyme, an anti-estrogen agent, a receptor tyrosine kinase inhibitor, a kinase inhibitor, a cell cycle inhibitor, a DNA, RNA or protein synthesis inhibitor, a RAS inhibitor or a combination thereof.

29. The method of claim 24, wherein the subject is a human.

30. A solution comprising an effective concentration of the isolated mAb or an antigen-binding fragment thereof of claim 1, wherein the solution is blood plasma in a subject.
